(19) United States
(12) Patent Application Publication
(10) Pub. No.: US 2024/0249808 A1
(43) Pub. Date: Jul. 25, 2024

(54) Physiologically Based Toxicokinetic (PBTK) Modeling for Implant Toxicology

(71) Applicant: Cyprus University of Technology, Limassol (CY)

(72) Inventors: Konstantinos Kapnisis, Limassol (CY); Pavlos S. Stephanou, Nicosia (CY); Andreas Anayiotos, Nicosia (CY)

(21) Appl. No.: 18/290,417

(22) PCT Filed: May 27, 2022
(86) PCT No.: PCT/EP2022/064468

§ 371 Date: Nov. 13, 2023

Related U.S. Application Data
(63) Continuation of application No. 63/193,947, filed on May 27, 2021.

(51) Int. Cl. G16H 20/13; G16C 20/30; G16C 20/70; G16C 60/00; G16H 20/40

(52) U.S. Cl. CPC G16H 20/13; G16C 20/30; G16C 20/70; G16C 60/00; G16H 20/40

(57) ABSTRACT

A method for toxicological risk assessment of a medical implant device, the device for implant into the body of a mammal, comprising: a) inputting device-specific data into a physiologically based toxicokinetic (PBTK) model for the distribution of a substance in the mammal into which the medical implant device is to be implanted, wherein the device-specific data are parameters characterising the medical implant device and/or its behaviour; b) determining, from the PBTK model, a time dependent function for the concentration level of the substance in a tissue, organ, biofluid and/or excreta of the mammal; c) determining, using the time dependent function for the concentration level of the substance, the maximum of the concentration level during a predetermined time interval; d) comparing the maximum of the concentration level with a predefined exposure threshold for the tissue, organ, biofluid and/or excreta; wherein if the maximum of the concentration level exceeds or is equal to the predefined exposure threshold, then identifying the medical implant device characterised by the device-specific data as having an unsatisfactory toxicological risk; or if the maximum of the concentration level is less than the predefined exposure threshold then identifying the medical implant device characterised by the device-specific data as having a satisfactory toxicological risk.

### CROSS-REFERENCE TO RELATED APPLICATIONS

This application claims the benefit of U.S. Provisional Patent Application No. 63/193,947, filed May 27, 2021, and titled “Physiologically Based Pharmacokinetic (PBPK) Modeling for Implant Toxicology”, which is incorporated by reference herein in its entirety.

### FIELD OF THE INVENTION

The present invention relates to a method for toxicological risk assessment of a medical implant device using a physiologically based toxicokinetic model (PBTK). The method may assess whether the release of one or more substances from a medical implant device is below a permissible exposure limit (PEL) or predefined exposure threshold. Also described is a method for optimisation or design of a medical implant device to identify preferred device specific data (device design parameters and/or implantation site-related parameters) for a proposed medical implant device.

### BACKGROUND TO THE INVENTION

Medical implants experience wear and corrosion due to the mechanical and biochemical environment at the implantation site. In vivo corrosion is a limiting design constraint on implant longevity as it presents the risk of deterioration of the material mechanical properties that could predispose fatigue fracture or trigger the release of debris. Many of the materials used (metals, polymers, ceramics, composite and/or hybrid materials, etc.) contain high levels of potentially toxic ions which, if leached, contaminate the surrounding tissue and/or the bloodstream and may cause several adverse local as well as systemic effects. Numerous studies reported different modes of toxicity, ranging from an allergic reaction to nephrotoxicity and carcinogenicity, for many compounds at varying doses (see Barchowsky. Systemic and Immune Toxicity of Implanted Materials. Biomaterials Science (Fourth Edition), An Introduction to Materials in Medicine 2020, pg. 791-799).

The corrosion resistance of implant materials results from optimal surface treatments during manufacturing, such as electropolishing, acid passivation, and protective coatings. Although the passive film insulates the bulk material from the corrosive physiologic fluids, any break or defect in the film increases the risk for corrosion. Biocorrosion may enhance the inflammatory reaction, depress the immune system, and facilitate peri-implant bacterial growth. Especially metal-on-metal coupling, e.g., orthopedic fixation devices, introduces additional biologic risks associated with increased degradation products of prosthetic materials. Additionally, inflammatory-cell reaction and peri-implant osteolysis induced by polyethylene particles represent the main problem in knee and hip joint implants with metal-on-polymer coupling. Even though ceramic-on-ceramic coupling represents a good alternative, a serum ion increase is observed probably due to the fretting at the head/neck connection (see J Biomed Mater Res B Appl Biomater. 2017 October; 105(7): 2162-2173; and, Clin. Cases Miner Bone Metab. 2013 January;10(1):34-40).

For cardiovascular devices, the effects of constraining the device in a delivery system (i.e., radial compression) and then releasing the constraint during deployment may damage the protective layer. Although metal alloys may be able to sustain this large forward and reverse mechanical strain excursion, the non-superelastic oxide layer may crack under large strains, creating a conduit for exposure of the metal ion-rich phases to the in vivo environment which will result in increased ion release and decreased resistance to pitting. In cardiovascular and endovascular stents, nanometer-thick regions of oxides are lost under complex in vivo conditions such as vessel tortuosity, high curvature, vascular wall stresses as well as blood flow wall shear stresses, and diffuse calcification (see J. Invasive Cardiol., vol. 22, no. 11, pp. 528-35, November 2010; and J. Biomed. Mater. Res. B. Appl. Biomater., vol. 95, no. 1, pp. 225-38, October 2010).

These conditions may compound their effects when two or more overlapping devices are deployed, a common clinical practice in interventional procedures especially in areas of branches and bifurcations or when treating long or recurrent lesions.

Medical devices are classified by government regulatory authorities, including the U.S. Food and Drug Administration (FDA), the Medical Devices Bureau of Health Canada, the European Commission on Health and Consumers (ECHC) and the Therapeutic Goods Administration (TGA). Any medical device approved by the FDA Center for Devices and Radiological Health (CDRH) is classified as either Class I, II, or Ill depending on the new device's risk, invasiveness, and impact on the patient's overall health. The FDA defines Class III devices as products which “usually sustain or support life, are implanted or present a potential unreasonable risk of illness or injury.” This classification is generally extended to implants, smart medical devices, and life support systems. Implantable devices are routinely used to address many different conditions in almost every medical specialty and may be temporary (designed to be removed or replaced) while others are meant to be permanent. The term “implant” refers to a wide variety of medical devices including but not limited to:
Cardiovascular implants (coronary stents, artificial heart valves, septal occluders, aortic aneurysm implants, artificial heart, cardiac pacemakers, implantable cardiac defibrillators (ICDs), cardiac resynchronization therapy (CRT) devices, etc.)
Orthopedic implants (joint, bone, or cartilage replacements)
Neurologic implants (stents and coils for neurovascular interventions, blocks used in vertebral repairs, tents, cerebral diversion device and other implants for cerebral aneurysms, deep brain stimulation, cochlear implants)
Oral and dental implants
Peripheral endovascular and biliary stents
Urogenital implants
Intrauterine implants
Breast Implants
Interocular implants
Implantable insulin pumps

Given the large number of devices implanted annually, investigating their behaviour in the highly corrosive physiologic environment is significant. There is currently no recognized standard test method for ion and/or particle release from medical implants, thus it is typically estimated through in vitro immersion tests. However, these approximations are not representative of the release rate and corrosion level following implantation in the body. Studies of implant retrievals from cadavers, indicate significant corrosion damage not consistent with the original estimates of the manufacturers (see Brown et al., Toxicity of Metals Released from Implanted Medical Devices. Handbook on the Toxicology of Metals (Fourth Edition) vol. I, 2015, pg. 113-122). Concentrations of different ions in serum and urine have been reported for healthy adults in many studies. Yet, there is limited information about serum ion levels and especially their distribution in tissues following the implantation of medical devices. Collection of human data on toxicological response has many challenges: timeframes associated with the complex physiology are varying and the data is heterogeneous (due to material and design characteristics of different devices). There is also the difficulty and cost implications of bringing patients back to the clinic electively for non-routine follow-up examinations. As such, there is a requirement within industry for an effective and accurate method for investigating the release of substances from an implantable device prior to implantation into a patient.

In the past, manufacturers and organizations had to adhere to relatively limited standards and regulations; hence, it was possible for companies to build their in-house testing and inspection capabilities as required. Recently, the standards pertaining to human/user safety have increased and additional studies are required that can be difficult, prohibitively costly, and time-consuming. This is a huge barrier to innovation by medical device manufacturers and a restraining factor for the growth of the medical device testing market. With introduction of its latest Guideline (ISO 10993-18:2020 Biological evaluation of medical devices), the International Organization for Standardization solidified and stressed the importance of a proper Toxicological Risk Assessment by implant manufacturers, which is now considered a prerequisite to biocompatibility testing.

A recent FDA report (FDA CDRH, Biological Responses to Metal Implants. September 2019) suggests that modelling and simulation tools during the implant design stage represent a promising and relatively easy way to potentially establish such quantitative relationships. Hence, there is currently a clear need for a reliable Toxicological Risk Assessment tool, early in the device design cycle, that will enable medical implant designers/manufacturers to plan the iteration process of a robust device development.

Many known approaches are based on pharmacokinetic profiles of a xenobiotic disposition in a mammalian tissue. For example, Fenetteau (US20080221847A1) discloses a physiologically based pharmacokinetic model that takes mammalian-specific data of transporter properties that comprise in-vivo estimated diffusion values through mammalian tissue. Rostami-Hodjegan (WO2021061804A1) discloses a process of establishing a virtual physiologically based pharmacokinetic model in a population comprised of a plurality of individual subjects that has been exposed or may be exposed to a xenobiotic molecule. Willmann and Schmitt (U.S. Pat. No. 7,539,607B2) disclose a method for calculating a pharmacokinetic behavior of a chemical substance in an insect based on at least one physicochemical property of said chemical substance. Grass et al (U.S. Pat. No. 6,542,858B1) disclose a method of predicting a pharmacokinetic property of a compound in a first anatomical segment of a mammalian system from a pharmacokinetic property of the compound in a second anatomical segment of the mammalian system. In fact, several patent applications disclose various methods of predicting diffusion of some xenobiotic substance in a mammalian system.

Modelling of the kinetics of substances in the human body is well known. For example, Sunderman et al. (Proc. Soc. Exp. Biol. Med. 191, 5-11 (1989)) disclosed a mathematical modelling of the kinetics of nickel absorption, distribution, and elimination was performed in healthy human volunteers who ingested NiSO in drinking water and in food. This was a compartmental model of nickel metabolism and considered tissue and biofluids.

Saylor et al. (Regul. Toxicol. Pharmacol. 80, 1-8 (2016)) disclose a biokinetic model for nickel released from cardiovascular devices. This model links the rate of in-vitro nickel release from a cardiovascular device to serum nickel concentrations to estimate the rate and extent of in-vivo nickel release from an implanted device. In the same paper, Saylor et al. (2016) sought to address the problem that while nickel release from implantable devices is typically characterized through the use of in-vitro immersion tests, it is unclear if the rate at which nickel is released from a device during in-vitro testing is representative of the release rate following implantation in the body. To address this uncertainty, they developed a biokinetic model that combines a traditional toxicokinetic compartment model with a physics-based model to estimate nickel release from an implanted device. This model links the rate of in-vitro nickel release from a cardiovascular device to serum nickel concentrations, to estimate the rate and extent of in-vivo nickel release from an implanted device. The biokinetic model for nickel released from cardiovascular devices combines a compartmental model for absorption, distribution, and excretion of nickel with a biphasic diffusion model for the release of nickel from the device. This model can be useful in biomonitoring applications. Subsequent work of Saylor et al. (Acta Biomaterialia 70 (2018) 304-314) considered specific device characteristics such as surface character and surface area on the maximum nickel concentration in local tissue and in serum following implantation of a cardiovascular device comprised of nitinol wire.

Kapnisis et al. (Summer Biomechanics, Bioengineering and Biotransport Conference, 2020, 17-20 Jun.) have discussed local diffusion and biotransport phenomena, using in silico toxicokinetic model to determine concentrations of released nickel ions from cardiovascular stents into the body over time. A set of equations consider different organs based on the compartmental pharmacokinetic model of Sunderman et al. and the work of Saylor et al. (2016). Kapnisis et al. argued that the model presented could be parametrized using data from in vitro immersion tests to predict the biodistribution of nickel released from implanted devices.

While prior art provides means of modelling the diffusion of substances in mammalian compartments, and even considers the release of substances from some implantable devices, it does not provide a practical and cost-effective way of guiding the optimal design of implantable devices to ensure compliance with permissible exposure limit of different mammalian compartments to concentrations of released substances including intervals of confidence.

There is therefore the need for a method and an apparatus that overcome the above-mentioned limitations so that device manufacturers can optimize device design and ensure that the release of substances will remain below acceptable thresholds.

### SUMMARY OF THE INVENTION

The present invention addresses the significant and continuing health problem of toxic contamination caused by medical device materials (stable or biodegradable) following implantation in patients. Manufacturers require an accurate estimate of the degree of corrosion and ion and/or particle release of their specific products during the initial design stage to optimize the device characteristics and to accelerate the completion of regulatory procedures.

The invention provides an in-silico tool and utilizes a physiologically based toxicokinetic (PBTK) model and a computer-implemented machine-learning method to estimate the concentration-time profiles, along with confidence intervals, of the released ions and/or particles from implantable devices (stable or biodegradable) in tissue and biofluid compartments of a mammalian system for the purpose of optimizing the design of medical implants to ensure that the release of one or more substances from said medical implants is below permissible exposure limit (or predefined exposure threshold).

The method for toxicological risk assessment of a medical implant device, the device for implant into the body of a mammal, comprising:
a) inputting device-specific data into a physiologically based toxicokinetic (PBTK) model for the distribution of a substance in the mammal into which the medical implant device is to be implanted, wherein the device-specific data characterises the medical implant device and/or its behaviour at a specific site of implantation;
b) determining, from the PBTK model, a time dependent function for the concentration level of the substance in a tissue, organ, biofluid and/or excreta of the mammal;
c) determining, using the time dependent function for the concentration level of the substance, the maximum of the concentration level during a predetermined time interval;
d) comparing the maximum of the concentration level with a predefined exposure threshold for the tissue, organ, biofluid and/or excreta;
wherein if the maximum of the concentration level exceeds or is equal to the predefined exposure threshold, then identifying the medical implant device characterised by the device-specific data as having an unsatisfactory toxicological risk; or
if the maximum of the concentration level is less than the predefined exposure threshold then identifying the medical implant device characterised by the device-specific data as having a satisfactory toxicological risk.

Based on model estimations, device parameters (device-specific data, being both or either implantation site-related parameters and device design parameters (e.g. total active surface area, geometric design, method of fabrication and processing, surface treatment and finishing process, physicochemical surface modification with or without overcoating (stable or biodegradable), bulk material and/or overcoating degradation rate)) can be perturbed around an initial value as part of the development process of implantable medical devices for optimizing the design in consideration of substance release. The predicted toxicokinetic profiles of the interested substance in a mammalian tissue or biofluid may be utilized by medical implant manufacturers, implant R&D companies, the medical device testing industry, or any other entity to optimize their iterative design process and minimize their development costs. Research Institutes and Universities doing research on novel designs of medical implants may also use the prognostic tool to facilitate enhancements in material properties and design characteristics. The increasing need for verification and validation for medical devices and the imposition of stringent government regulations to ensure that the products comply with the quality, safety, and performance standards, have led regulatory bodies worldwide to strongly recommend the use of modeling and simulation tools to support medical device submissions. The consistency and predictability of in silico models will eventually enable their use by physicians to improve decision-making in clinical practice.

According to the invention, a toxicokinetic compartment (i.e., a compartment that represents one of biofluids, tissues or organs, or excrements) is combined with a physics-based model that describes the transfer of the substance from the device to adjacent tissue and circulation, and the exchange between blood and the various tissues/organs. A physiologically based toxicokinetic (PBTK) model results, which includes a detailed set of constitutive equations relating diffusion, absorption, distribution, and excretion variables as well as the initial and boundary conditions for leaching and biokinetics.

Various input parameters may be extracted using: (a) cumulative release data derived from in-vitro corrosion or degradation tests (static and/or dynamic), and (b) in-situ implantation studies to establish the time-dependent concentration of the released ions/particles in tissues and biofluids. It is also an object to provide an improved method (using non-linear curve-fitting algorithms) for optimizing the calculation of the device-specific diffusion parameters and the mammalian-specific absorption, distribution and excretion properties (determined once for the specific device and mammal type).

The model-derived output predicts whether, for a specific proposed implant device design implanted into a mammalian body, permissible levels of a substance would be exceeded. Thus, the method indicates if the proposed implant device design would be toxicologically safe or unsafe (in other words, whether it would pose a satisfactory toxicological risk or an unsatisfactory toxicological risk, respectively). This enables the early screening of defective designs and the identification of device flaws before any biocompatibility experiments take place in vivo. Most importantly, it offers alternative approaches that promote the principles of the 3Rs (Replacement, Reduction and Refinement), that minimize the use of animals in biomaterial testing, and that facilitate a considerable reduction in time and design costs such as repetitive biocompatibility and preclinical animal experiments required in the regulatory approval procedure.

In addition, an in-silico tool is described which implements the described method. The in-silico tool may incorporate computer-readable components including: (1) an input/output system; (2) a physiologic-based simulation model of the mammalian system of interest; and (3) a simulation engine having a differential equation solver.

The following numbered clauses provide further illustrative examples of the described method, in which the PBTK model is referred to as a Physiologically Based Pharmacokinetic (PBPK) model:

1. A computer-implemented machine learning method based on a PBPK model for developing simulated toxicokinetic profile of a substance, said substance being released (or leached) from a device into a mammalian tissue or biofluid, said method comprising:
a) inputting mammalian-specific data into said PBPK model, wherein said mammalian-specific data comprise total blood volume per body weight, volumetric urine and fecal output rate, respiration rate;
b) inputting substance-specific data into said PBPK model, wherein said substance-specific data comprise substance concentration in the average daily food and water intake, substance concentration average daily inhaled;
c) inputting device-specific cumulative release profile data of said substance into said PBPK model following in vitro corrosion or degradation tests (static and/or dynamic);
d) inputting data into said PBPK model from the concentration/time profile of said substance released in tissue or biofluid compartments of a mammalian system;
e) using computer means for initializing and solving the mathematical equations comprising said PBPK model.

2. The method according to clause 1, further comprising using said computer means to perform a successive fitting procedure to obtain the values of said PBPK model parameters that best describe the experimental data obtained from the in vivo study.

3. The method according to clause 2, further comprising using said computer means to store said PBPK model predicted data.

4 The method according to clause 2, further comprising using said computer means to perturb the value of at least one of said device-specific data.

5. The method according to clause 2, further comprising using said computer means to calculate anew said PBPK model predicted data.

6. The method according to clause 5, further comprising using said computer means to store the new PBPK model predicted data.

7 The method according to clause 5, further comprising using said computer means to repeat said perturbing and said storing the predicted data.

8. The method as claimed in clause 7, further comprising the production mode to generate one or more of the predicted data sets (concentration/time profile of said substance in a specific tissue or biofluid compartment of the mammalian system).

9. The method as claimed in clause 8, further comprising the production mode using the computer to store the one or more of the predicted data sets (concentration/time profile of said substance in a specific tissue or biofluid compartment of the mammalian system).

10. The method according to clause 9, further comprising processing and providing said predicted data for use in at least one of evaluation, manufacturing decision-making, design decision-making, decision-making pertaining to regulations, clinical decision-making related to the manufacturing, design or clinical use of said devices.

11. The method according to clause 1, further comprising the use of a mass spectrometry analysis apparatus.

12. The method according to clause 1, wherein said mammalian tissue is selected from at least one of the followings: peri-implant tissue, liver, kidneys, lungs, brain, gut and skin.

13. The method according to clause 1, wherein said mammalian biofluid is selected from at least one of the followings: blood serum, urine and sweat.

14. The method according to clause 13, wherein said substance is selected from the group comprising chemical ion, particle, particulate, a drug, an herbal medicine, a chemical organic or inorganic compound, said substance being released from any type of medical implant (stable or biodegradable).

15. The method according to clause 14, wherein said mammalian comprises any laboratory animal or human.

16. The method according to clause 15, wherein said mammalian-specific data further comprises physiological and anatomical properties of mammalian-related data and implantation site-related data.

17. The method according to clause 16, wherein said physiological and anatomical properties of said mammalian related data are selected from the group comprising the volume of tissues, substance composition in said mammalian tissues, total blood volume per body weight, volumetric urine and fecal output rate, and respiration rate.

18. The method according to clause 16, wherein said implantation site-related data of said mammalian-specific data are selected from the group comprising local biomechanical (geometric and loading parameters) and biochemical characteristics, hemorheological properties and hemodynamic parameters that describe the local and peripheral blood flow profile, etc.

19. The method according to clause 1, wherein said substance-specific data further comprises physiochemical properties of said substance-specific data.

20. The method according to clause 1, wherein said device-specific data comprise various properties related to said device, including total active surface area, geometric design, method of fabrication and processing, surface treatment and finishing process, physicochemical surface modification with or without overcoating (stable or biodegradable), bulk material and/or overcoating degradation rate.

21. The method according to clause 1, wherein said simulated toxicokinetic profile of said substance comprises a predicted distribution profile of said substance in said mammalian tissue.

22. The method according to clause 1, wherein said simulated toxicokinetic profile of said substance comprises a predicted profile of the absorbed and desorbed quantity and rate of said substance in said mammalian tissue.

23. The method according to clause 1, wherein said simulated toxicokinetic profile of said substance comprises a predicted concentration profile of said substance as a function of time in said mammalian tissue.

24. The method according to clause 1, wherein said method further comprising imputing into said PBPK model simulated pharmacokinetic profile of said substance including an eliminated amount of said substance to renal, and fecal excretion, as well as excretion from the skin and fur/hair.

25. The method according to clause 1, further comprising using on said computer means of a model training process to train a machine learning system on said imputed data to predict said toxicokinetic profiles of said substance in said mammalian tissue or biofluid.

26. The method according to clause 1, wherein said pharmacokinetic profiles data of said substance include at least the local tissue adjacent to said device.

27. The method as in clause 25, further comprising said model training process on said computer means to calculate said concentration-time profiles of said substance in at least one of said mammalian tissue.

28. The method as in clause 25, further comprising said model training process on said computer means to store said calculated concentration-time profiles of said substance in at least one of said mammalian tissues.

29. The method according to clause 1, comprising the algorithms embedded in the software to carry out the method.

### BRIEF DESCRIPTION OF THE DRAWINGS

Examples of embodiments of the invention will now be described by referring to the accompanying drawings:

FIG. 1a is a schematic illustration of a mammalian system with various compartments, as well as intake and elimination of one or more substances;

FIG. 1b is a schematic illustration of a mammalian tissue with absorption and desorption of a substance;

FIGS. 1c and 1d are illustrative examples of indicative concentration curves and confidence intervals for the concentration of a substance in mammalian tissue and excrement over time as a result of an implanted device.

FIGS. 1e and 1f are illustrative examples of indicative concentration curves with confidence intervals for the concentration of a substance in mammalian tissue and excrement over time as a result of a first and a second implanted device;

FIG. 2 is a simplified representation of the model used by the method;

FIGS. 3a to 3c are further simplified representations of the use of the model according to the method;

FIG. 4a is a diagram illustrating the diffusion, absorption, distribution and excretion principles of the PBTK model;

FIG. 4b is a diagram illustrating the diffusion, absorption, distribution, and excretion principles of the PBTK model with two devices at a single implantation site;

FIG. 4c is a diagram illustrating the diffusion, absorption, distribution, and excretion principles of the PBTK model with two devices at different implantation sites;

FIG. 5 is a diagram illustrating the diffusion, absorption, distribution, and excretion principles of a coarse-grained version of the PBTK model with a single implanted device;

FIGS. 6a and 6b are diagrammatic illustrations of the process of estimating model parameters and the model-derived prognosis for a specific mammalian type according to an example of the method;

FIG. 7 is a diagrammatic representation of the method showing alternative means of fitting the model predictions against available experimental data;

FIG. 8a is an outline of example of mammalian-specific properties and data;

FIG. 8b is an outline of example of device-specific properties and data;

FIG. 9 is a flow diagram showing the steps of the described method for toxicological risk assessment of a medical implant device;

FIG. 10 shows plots of time dependent functions for the concentration level of a substance in different mammalian tissue and excreta, obtained from the described PBTK model; and

FIG. 11 is a schematic diagram showing elements of a system for carrying out the described method.

### DETAILED DESCRIPTION OF EMBODIMENTS

Implantable devices (medical implant devices) are routinely used to address many different conditions in almost every medical specialty. They may be temporary (designed to be implanted and then later removed or replaced) while others are meant to be implanted permanently. The term “implant” refers to a wide variety of medical devices such as those mentioned in the “Background to the invention” section above. Such medical devices can be implanted in various parts of the body, and in some cases more than one medical implant device may be implanted in the same body (within the same organ or tissue of a mammalian body, or in different locations in the body). One example of an implantable device is being a stent device for implant into a blood vessel.

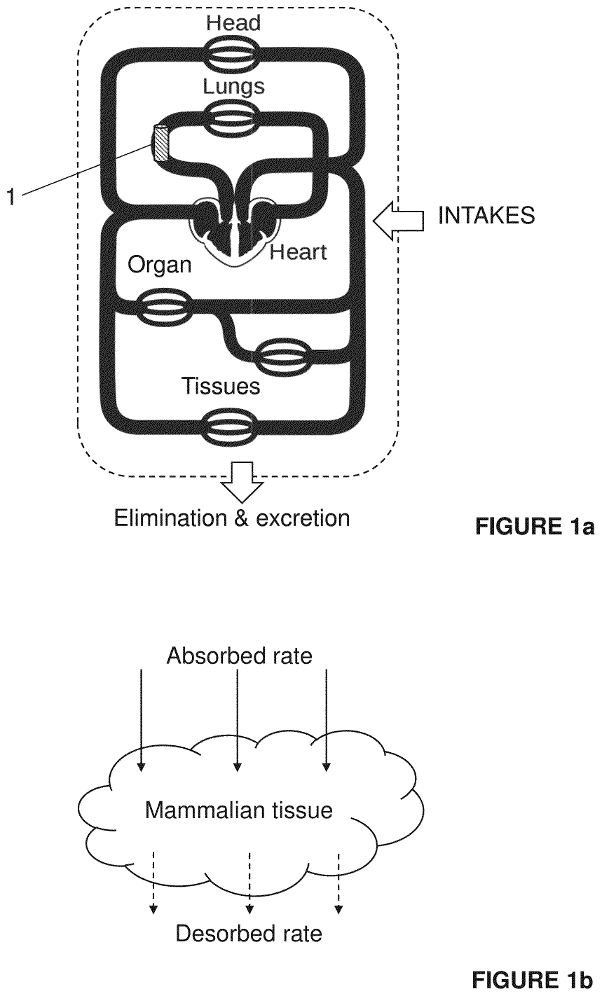

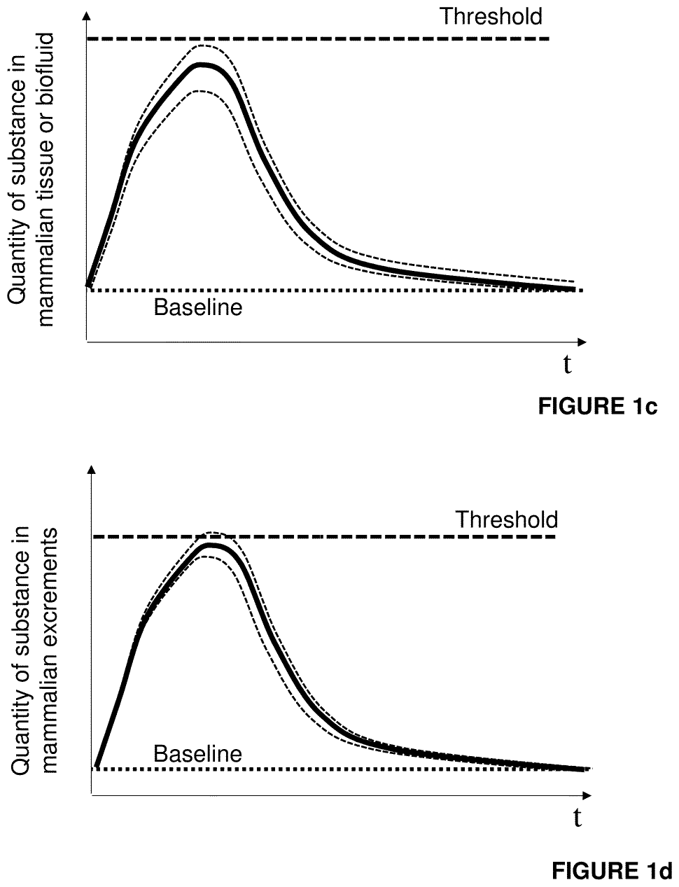

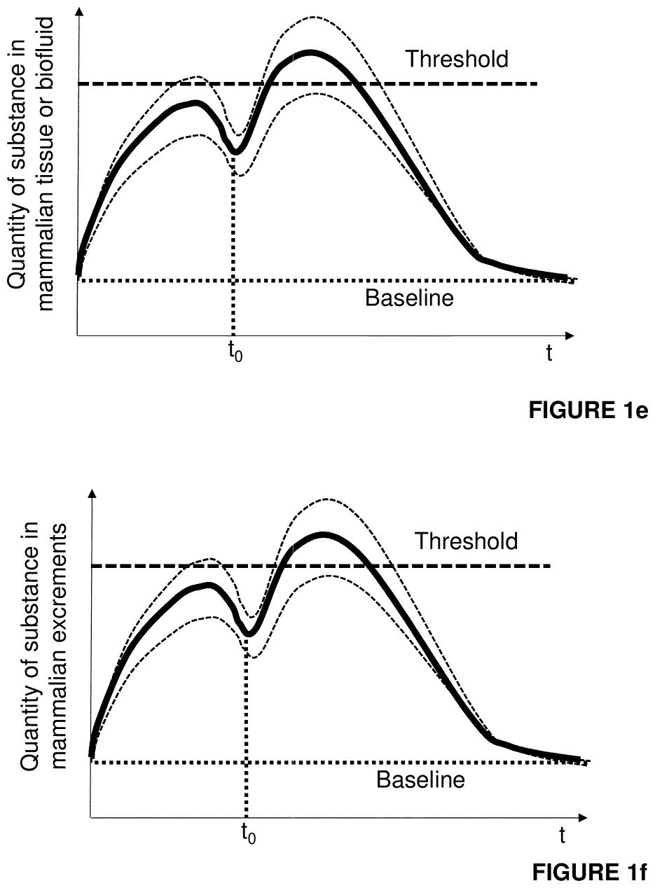

FIG. 1a is a schematic illustration of a mammalian system with various compartments, together with intake and elimination of substances. Ions, molecules or particles may be released by a medical implant device (1) implanted into the mammalian system. More specifically, the medical implant device is a stent implanted into a blood vessel between the heart and lungs, as one example. The medical implant device may release a foreign or contaminant substance (that is unintentional, and not the purpose of the medical implant device). After initial leaching from the device, transfer of the foreign or contaminant substance (being ions, molecules and/or particles) may take place via diffusion, absorption, distribution, and excretion so as to be distributed through the circulatory system to organs, tissues, and excretions. The foreign ions and/or particles may be transferred to tissues adjacent to the implanted device and to the bloodstream.

The local physiochemical environment plays an important role in the degradative performance of an implant. Implantable devices experience different degrees of wear and corrosion due to the mechanical and biochemical environment at the specific site of implantation. The complex and varying in vivo setting comprising dynamic geometries, cyclic and dynamic loading profiles, and harsh chemical conditions (chloride, dissolved oxygen and PH levels) can affect corrosion susceptibility that results from surface damage or a change in ion diffusion kinetics. Understanding how materials change or degrade in different environments is an important concept when speaking about potential host responses.

For cardiovascular devices, nanometer-thick regions of oxides are lost under complex in vivo conditions such as vessel tortuosity, high curvature, vascular wall stresses as well as blood flow wall shear stresses, and diffuse calcification. These conditions may compound their effects when two or more overlapping devices are deployed, which is a common clinical practice in interventional procedures especially in areas of branches and bifurcations or when treating long or recurrent lesions. For orthopedic devices, which are subjected to severe mechanical stresses, increased levels of metallic (namely, Ti—, Co— and Fe-based alloys) and polymeric (ultrahigh molecular weight polyethylene-UHMWPE, polyoxymethylene-POM, and poly(methyl methacrylate)-PMMA) particles were detected in synovial fluids following implantation of artificial hip and knee joints, which is attributed to the synergistic effect of fatigue corrosion and wear. Moreover, electrodynamic processes mediated by most implantable stimulators (used in cardiac, muscle, and neural devices) involve the transfer of electrons between an electrode and a solution, and this can accelerate material oxidation, corrosion, and ion release. It is, therefore, imperative to understand and analyze the multilevel interactions an implantable device is expected to experience in vivo in order to determine the testing necessary to establish a reasonable assurance of safety.

Any mammalian tissue absorbs and desorbs a substance at some rate. The absorption rate may be different from the desorption rate, in which case a substance concentration may increase or decrease in that particular tissue. FIG. 1b is a simple schematic illustration of this principle.

Biodegradation is an important factor in the design and selection of materials for service in vivo. Allergenic, toxic/cytotoxic or carcinogenic substances may be released into the body during the degradation processes. In addition, various corrosion mechanisms can lead to implant loosening and failure. Therefore, biomaterials are often required to be tested for corrosion and/or solubility before they are approved by regulatory organizations. Hence, the degradation behaviour of implant materials has been widely studied, in the framework of quality assurance, implant retrieval analysis, and failure analysis.

In general, degradation/corrosion is the uniform release of ions/particles over an exposed surface. For metals with surface oxides, it has been shown that the amount of metal ions released from the implant is dependent on the composition and structure of its oxide layer. Typically, the release of metal ions is greatest immediately after implantation and the release rate reduces over time. However, in cases where the implant's oxide layer is not protective, the release of metal ions may continue for longer durations and exhibit dramatic increases in release rate after implantation. For example, in cardiovascular devices such as stents, the implant may cause local inflammation during placement, but gradual tissue coverage associated with normal inflammation in conjunction with blood flow will present a dynamic physiological environment to the stent. If corrosion is occurring, the local inflammatory response can be aggravated. For polymer-coated esophageal stents, low pH environment is the norm due to the presence of local gastric acid. Dental implants can experience a wide range of pH change because of a patient's diet.

Mechanical interactions (fatigue, fretting, and wear) of implants can also affect corrosion susceptibility. Damage or disruption to the passive oxide layer can either weaken the protective layer or directly expose the underlying layers to corrosion. Debris from fretting and other wear mechanisms by themselves can have a significant effect on ion release rates even when the implant has a low ion release rate from general corrosion.

Animal studies are often used to evaluate the local tissue response in a relevant anatomical site under simulated clinical conditions. However, biocompatibility assessments are not typically designed to evaluate the biological response to mechanical failure and anatomical differences between humans and animal models limit quantitative correlation. Additional studies are required to evaluate the in vivo response to failure modes such as coating delamination (separation into layers) or the generation of wear particulates (e.g., subcutaneous implantation of material/device extracts and by-products).

For the case of one implanted device and one monitored substance of interest, a qualitative illustrative profile of the concentration of this substance is provided in FIGS. 1c and 1d. FIG. 1c shows a typical concentration profile curve (solid line) of a substance in a mammalian tissue or biofluid in which a single device is implanted. FIG. 1d shows a typical concentration profile curve in an excrement, the excrement being from the mammalian system after the implantation of the single device. Under normal device behavior, the mass distribution in tissues/biofluids conforms to exponential growth and decay curve. FIGS. 1c and 1d further show a toxicological threshold value (broad dashed line). The toxicological threshold may also be known as a predefined exposure threshold. The toxicological threshold represents the concentration of the given substance at which the substance may pose a risk to the health of the mammal into which the medical implant device is implanted. When designing the medical implant device, it is required to keep the concentration profile below the toxicological threshold value in all the tissue, organ, biofluid and the excrement compartments, at all times. In the particular examples represented in FIGS. 1c and 1d the concentration-time profiles always remain below the toxicological threshold, although the upper bound of the confidence interval exceeds the toxicological threshold in FIG. 1c (representing the substance concentration in the mammalian excrement).

Confidence intervals provide a means of assessing and reporting the precision of the concentration-time profile estimated by the PBTK model. They represent the uncertainty in the model predictions and allow the user to determine if they can expect similar results in a real mammal, in-vivo (for example, in preclinical studies). In some cases, the whole confidence interval may be required to be below the toxicological threshold in order to be considered to have satisfactory toxicological risk.

FIGS. 1e and 1f are examples of typical concentration curves for the concentration of a monitored substance of interest in mammalian tissue over time where both a first and a second device is implanted. FIG. 1e shows the typical concentration profile of the monitored substance after the implantation of two devices in a tissue or biofluid, and FIG. 1f shows the concentration profile of the monitored substance in excrement after the implantation of two devices. In this case, in which two devices are implanted, the qualitative concentration profile (as shown in in FIGS. 1e and 1f) will, in general, feature two distinct peaks. In particular, a first peak may be observed a short time after implant of the first device, and a second peak may be observed a short time after implant of the second device. The second peak may represent a cumulative concentration of substance released from both the first and the second implant, and may represent an increased concentration compared to the peak from the first or single implant alone. The model can be applied when there are multiple devices (same or different type) implanted at the same or different sites (simultaneously or with a time lag (months, years, other)) or when the single device or various parts comprising a device (e.g. orthotopic implants) degrade at different time rates (due to mechanical failure or biochemically-induced biodegradation). This is a common clinical practice in interventional cardiology procedures especially in areas of branches and bifurcations or when treating long or recurrent lesions All the aforementioned examples, can be considered via the use of a particular time-dependent expression for the release rate, as obtained from appropriate in vitro testing setups simulating the specific in vivo environment of each particular device.

Referring to FIGS. 1e and 1f, the first device is implanted at time t=0 and the second device at time t=t₀. It can be observed that a first peak and subsequent reduction in concentration of the substance (in both the tissue or biofluid (FIG. 1e) or in the excrement (FIG. 1f)) is observed due to the release of substance from the first implanted device. Then, after time t=t₀, a second peak and subsequent reduction in the concentration of the substance is observed due to the release of the substance from the second implanted device. The second peak includes a cumulative concentration representative of the release of substance from the second and the first implanted device for a given time. In this example, taking into account accumulation of the substance, the second peak reaches a higher maximum concentration than the first peak. The characteristics of the implanted devices will influence the peak maximum concentration.

The behavior shown in FIGS. 1e and 1f, is similar to that observed when different parts of one implant device degrade at different times (for instance, one part degrading immediately after implant, and a second part degrading after time t=t₀). Model behavior/prediction is also comparable (to the case of two implanted devices) when rapid degradation starts (releasing a large dose of substance) such as in the case of pitting on the surface of an implanted device, thereby exposing a surface of a different material underneath. This type of degradation can result in a peak in concentration of a substance being observed a given time interval after implantation, and in some cases after an initial peak observed immediately after implantation. These scenarios can result in concentration curves comparable to the response after implantation of multiple devices at the original (or different) implantation site, simultaneously or with a time delay.

Similar to FIGS. 1c and 1d, a toxicological threshold value (broad dashed line) is shown in FIGS. 1e and 1f. It can be seen that, in this example, the second peak in concentration (or quantity of the substance) exceeds the toxicological threshold. This indicates a risk to the health of the mammal into which the device is implanted. The first peak remains below the toxicological threshold, although the upper bound of the confidence levels exceeds the threshold for both peaks. In any case, it can be seen that the interplay of different, multiple implanted devices must be considered to avoid exceeding the toxicological threshold overall. To avoid risks to a patient, it is important for a healthcare provider to accurately predict the cumulative substance concentration that might arise from implantation of multiple devices.

A baseline, shown in FIGS. 1c to 1f, represents the amount of the substance that is either inhaled or received through diet and environment (in other words, the amount of the substance within the tissue or excreta but that is not originating from the device or devices(s)). It may be, in some cases, zero, very small or have a significant value.

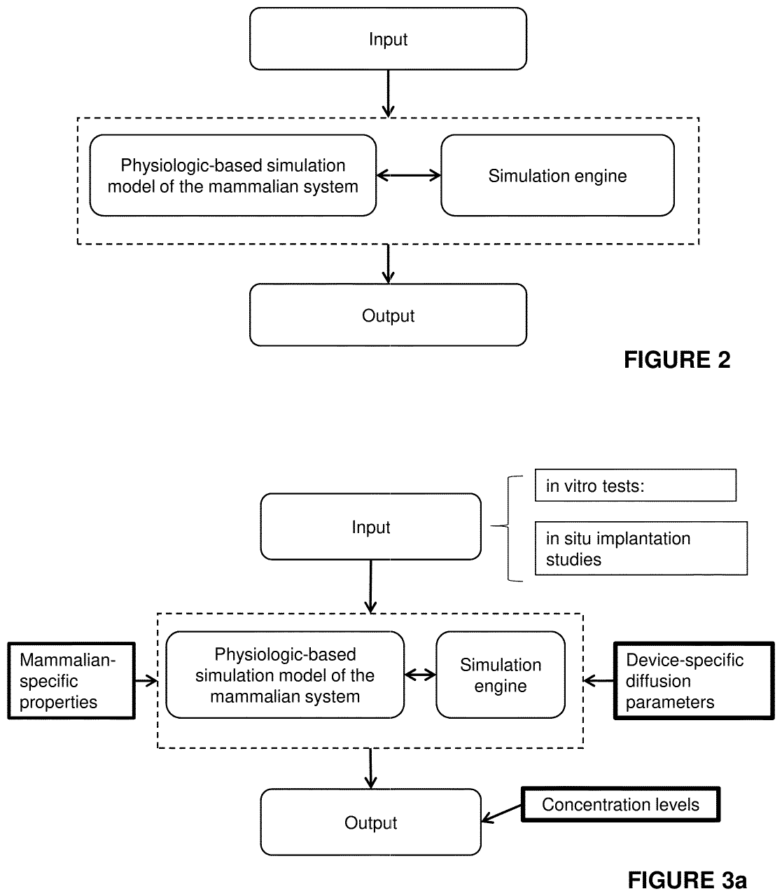

FIG. 2 is a simplified representation of the model used by the method. The method provides an in-silico tool and it relates to a PBTK model and a computer-implemented machine-learning method to estimate concentration-time profiles of the released ions and/or particles from medical implants (stable or biodegradable) in tissue and biofluid compartments of a mammalian system.

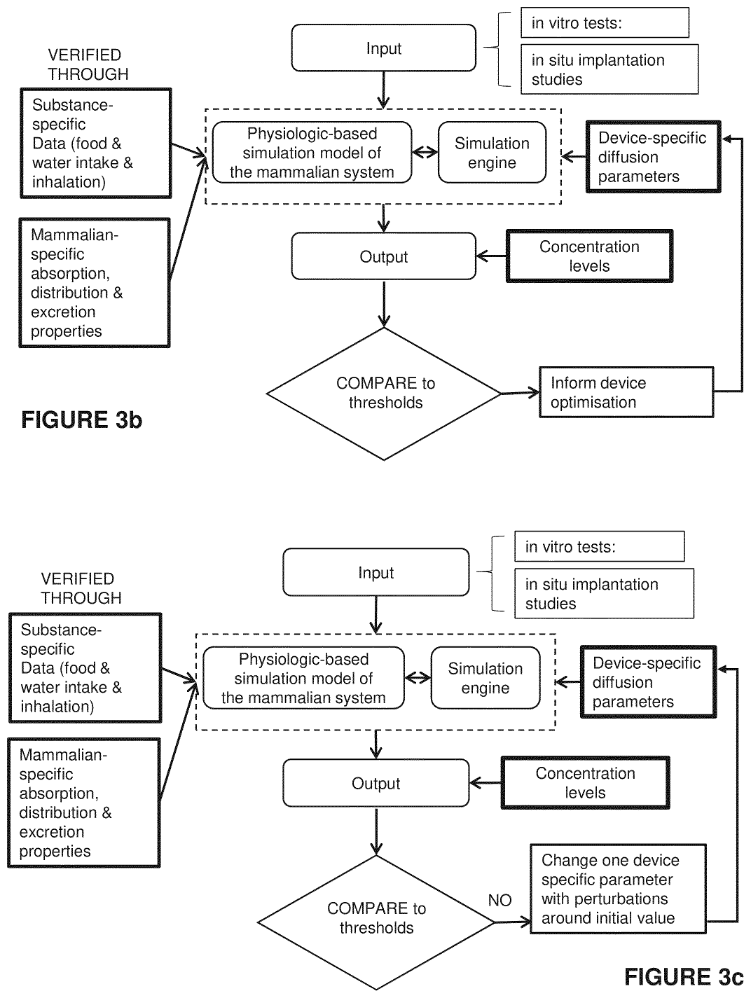

FIGS. 3a to 3c are simplified representations of the utilization of the model according to the method. As a first aspect, a toxicokinetic compartment model is combined with a physics-based model to describe the transfer of the substance from the device to adjacent tissue and circulation, and the exchange of the substance between blood and the various tissues/organs. The model relies on a detailed set of constitutive equations including diffusion, absorption, distribution, and excretion variables as well as initial and boundary conditions for leaching and biokinetics. The computer-implemented method may be carried out in a system comprising components including: (1) an input/output system; (2) a physiologic-based simulation model of the mammalian system of interest; and (3) a simulation engine having a differential equation solver.

FIGS. 3a to 3c progressively illustrate different aspects of the method. In FIG. 3a inputs, that are in-vitro tests and in-situ implantation studies, are used by the simulation model and simulation engine to produce outputs that refer to anticipated concentration levels of substances. In-vitro tests provide data on corrosion or other degradation of implantation devices. In-situ implantation studies provide time-dependent ion concentration data in mammalian tissues, when the model input substance used is ion concentration. These mammalian tissues may be for a mammal other than the mammal where the device is implanted in actual clinical use. For example, the in-situ implantation studies may be providing data from implantation in rats, while the device is ultimately intended for human use.

In FIG. 3b, the model is further informed by substance-specific data, and by mammalian-specific absorption, distribution, and excretion properties. For example, substance-specific data may refer to intake of water, food, or breathing rates. Mammalian-specific absorption excretion properties may refer to urination or sweating. The outputs of the simulation in the form of concentration levels, are compared to relevant thresholds. Levels above allowable thresholds require that the system performs a device optimisation process.

The referred thresholds (the toxicological threshold value, or predefined exposure threshold) are generally defined by the scientific, medical community and by regulatory authorities. For example, the FDA and the scientific and healthcare community will need to consider and address several individual and related challenges pertaining to adverse effects associated with implantable devices. Biocompatibility endpoints described in the CDRH Biocompatibility Guidance (Guidance for Industry and Food and Drug Administration Staff: Use of International Standard ISO 10993-1, “Biological evaluation of medical devices—Part 1: Evaluation and testing within a risk management process”) which could be impacted by substances in, on, or from an implantable device include: cytotoxicity, sensitization, irritation or intracutaneous reactivity, acute, subchronic and chronic systemic toxicity, material-mediated pyrogenicity, genotoxicity, implantation, hemocompatibility (hemolysis, complement activation, thrombosis), carcinogenicity, reproductive or developmental toxicity, and biodegradation (for absorbable materials).

Local toxicity refers to concentration levels (and subsequent adverse effects) at sites local to the implanted device. In comparison, systemic toxicity refers to adverse effects (other than systemic sensitization, genotoxicity, and carcinogenicity) that occur in tissues other than those at the site of local contact between the body and the device. The development of systemic toxic effects typically requires the release of chemical compounds from the device and the distribution of these compounds to distant target tissue sites where deleterious effects are produced. Organ-specific accumulations of certain ions together with the simultaneous ion-specific excretion rates from the body could lead to the establishment of elevated concentrations of specific alloy elements of implants. These include elevations of metallic content in tissue (at both local and remote sites, e.g., kidney and liver) and of metal-bearing ion concentrations in serum and urine. This could upset the overall balance established by physiological tolerance to toxicity. Systemic toxicity is included as a recommended endpoint in the biological evaluation of devices depending on the nature and duration of device contact with the patient (CDRH Biocompatibility Guidance; FDA).

Although local and systemic responses are known to manifest in patients with implantable devices, it remains unclear if a substance allergy is a cause or consequence of device failure. While assays to detect delayed Type IV hypersensitivity exist, whether patients should be screened for specific hypersensitivity prior to implantation remains subject to debate and an important issue to resolve. Moreover, recommended standards to assess the potential for adverse systemic effects from the release of chemical compounds from a device have their drawbacks. For example, ISO 10993-11 does not account for the fact that target organ toxicities can occur without changes in body weight and “clinical observations.” Additionally, chemical characterization/risk assessment using ISO 10993-1 (International Standard ISO 10993-1, “Biological evaluation of medical devices—Part 1: Evaluation and testing within a risk management process”. Guidance for Industry and Food and Drug Administration Staff. SEPTEMBER 2020) and the CDRH Biocompatibility Guidance can only be used if toxicity data are available for particles with the exact physical-chemical properties as those released from the device. Furthermore, sensitization or hypersensitivity is not used as an endpoint for chemical characterization/risk assessment. Limitations in biocompatibility assessments thus present unique challenges in the premarket evaluation of the device.

To assess the biocompatibility of an implantable device, the FDA recommends following the Biocompatibility Guidance (Guidance for Industry and Food and Drug Administration Staff: Use of International Standard ISO 10993-1, “Biological evaluation of medical devices—Part 1: Evaluation and testing within a risk management process”). This guidance identifies the types of biocompatibility assessments that should be considered and recommendations regarding how to conduct related tests. In addition to in vitro release testing, a risk assessment should be performed to compare the amount of ion released from the device to a Tolerable Intake (TI) value for the specific substance. A TI value is defined in the ISO 10993-17:2002/(R) 2012 standard “Biological evaluation of medical devices—Part 17: Establishment of allowable limits for leachable substances” as an “estimate of the average daily intake of a substance over a specified time period, on the basis of body mass, that is considered to be without appreciable harm to health”.

The toxicity of an elemental impurity is related to its extent of exposure (bioavailability). TI values have been determined for various elemental impurities of interest for three routes of administration: oral, parenteral, and inhalational. These limits are based on chronic exposure. For adverse systemic effects that may occur following prolonged or permanent patient exposure to a specific substance, except for hypersensitivity, CDRH recommends a TI value for parenteral (non-oral) exposure. This value is based on systemic toxicity data from experimental animals following administration by parenteral routes of exposure (e.g., intravenous, intraperitoneal) and derived using the approach outlined in the ISO 10993-17 standard (e.g., for nickel TI=0.5 μg/kg/day). It is important to note that the TI values are not intended to be protective for local effects (e.g., necrosis, inflammation, irritation) that may result from ion release from an implant into tissues surrounding the implant.

Harmful responses are often the result of device, biomaterial, and patient-related factors. Individual patient susceptibility likely plays an important role in the outcome, raising questions about how an implant recipient's immune system may respond to the presence of a substance in/from the device and to what degree, if any, that response may produce clinically meaningful signs, symptoms, or adverse outcomes. Special populations which may be more susceptible to specific foreign substances are not been identified. Personalized prognosis, prevention, and treatment strategies require that permissible tolerable intake levels are examined for specific subsets of the general population. Age, sex, race, ethnicity, and health status-related differences can be found in many areas of biomedical research and clinical medicine. For instance, there is evidence that sex differences in immune responses are responsible for the increased rate of failure in metal implants, e.g., orthopedic, which is consistent with previous studies demonstrating a significantly higher rate of self-reported cutaneous metal sensitivity among females.

The main challenge in both pre- and post-market phases of regulatory review is the lack of adequate study endpoints and prognostic tools which reliably predict clinical responses. The threshold for detecting subtle but consequential biological responses which may constitute signals in post-market surveillance systems needs to be determined. Currently, it is extremely difficult to determine whether symptoms are related to the implanted device or other causes. Predictive assessment of the proinflammatory potential and subsequent tissue remodeling remains a major challenge affecting real-world performance of implantable devices and biomaterials. Diagnostic and prognostic methods or other mitigation strategies which may predict or reduce the likelihood or severity of a patient's response are urgently needed. An effective screening tool for implants would strike an optimal balance between sensitivity (to enable detection before exceeding permissible tolerable intake) and robustness (to promote confidence that the prediction is valid).

The claimed invention can be utilized to interpret biomonitoring data but also serve as the basis for reverse dosimetry. If a level of release from the device associated with toxicological concern is established, the resulting critical tissue and biofluid concentrations can be specified. Through monitoring these levels, it is possible to assess whether the release from a comparable device is likely exceeding levels associated with toxicological concern. Patient- and organ/biofluid-specific tolerable intake values can be determined through computer-implemented machine-learning methods. The model-derived output will determine if permissible levels are exceeded and decide if the implant design is toxicologically safe or unsafe.

FIG. 3c illustrates a method to optimize device-specific data (being implantation site-related parameters and/or device design parameters) so that substance concentration levels can be brought below accepted thresholds.

There may be different thresholds applicable to the general population and to specific patient groups. Manufacturers themselves may choose to apply more stringent thresholds for specific patient groups, for example diabetics. The design of medical implants may be optimized for the general population or for specific patient groups.

The device-specific data (e.g., device design parameters and/or implantation site-related parameters) can be perturbed or adjusted to obtain the optimum solution (i.e., concentrations below the toxicological threshold). Perturbation of the device parameters may include changing the total active surface area, geometric design, method of fabrication and processing, surface treatment and finishing process, physicochemical surface modification with or without overcoating (stable or biodegradable), bulk material and/or overcoating degradation rate. The model predictions can also be utilized for critical decision-making in clinical practice such as optimizing the implantation protocol or site of implantation to enable short- and long-term stability of the device(s). This can be achieved by selecting the appropriate expression and parameter values for cumulative amount of substance released from the device and thus the release rate of the substance from the device, that has been obtained through the customized in vitro experiments simulating the specific in vivo environment. This in turn enables for the more accurate determination of the model biokinetic parameters associated with the local tissue release.

Each and every design parameter can be perturbed and consequently alter the mass of substance released from the device, M_{d}(t), and the rate of substance released from the device, \dot{M}_{d}(t)=dM_{d}/dt. Accordingly, a function describing the rate of release of the substance from the device to local tissue and blood stream (including blood serum) represents a device-specific data. The function describing the rate of release of the substance from the device to local tissue and blood stream may be obtained by in-vitro measurements (following experimental, theoretical or computational tests) for a given medical implant device. For instance, this can be carried out through in vitro immersion (static or dynamic) tests that provide the device-specific cumulative release profile data. Using a standard non-linear curve fitting algorithm, the M_{d}(t) equation is solved for the best fit of the unknown material quantities for each device that is characterized. Because this is time-consuming and requires significant experimentation, key design parameters may be selected in priority for such perturbations. Such selection may take place by various techniques taking into consideration physiological constraints, manufacturability and cost considerations.

Alternatively or additionally, the function describing the rate of release of the substance from the device to local tissue and blood stream is a function of one or more device design parameters. In other words, the device-specific data is a function representing a model or simulation of the rate of release of the substance from the device to local tissue and blood stream. One or more device design parameters may be modified, and then the new device specific data fed into the model to identify the effect of the modified design parameter on the resultant concentration of substance in a specific tissue or excreta. Perturbation of the device-specific data (device design parameters and/or implantation site-related parameters) may also be carried out via the formal application of a Perturbation Theory Machine Learning (PTML) model.

The model-derived output determines if permissible levels are exceeded and decides if the implant design is toxicologically safe or unsafe. This enables for the early screening of defective designs and the identification of device flaws before the biocompatibility experiments. Most importantly, it offers alternative approaches that promote the principles of the 3Rs (Replacement, Reduction and Refinement) and minimize the use of animals in biomaterial testing and facilitate a considerable reduction in time and design costs such as repetitive biocompatibility and preclinical animal experiments required in the regulatory approval procedure.

The in-silico tool and methods of the invention can also be utilized to interpret ion biomonitoring but also serve as the basis for reverse dosimetry. If a level of release from the device associated with toxicological concern is established, the resulting critical serum (blood) and urine concentrations can be specified. Through monitoring these levels, it is possible to assess whether the release from a comparable device is likely exceeding levels associated with toxicological concern.

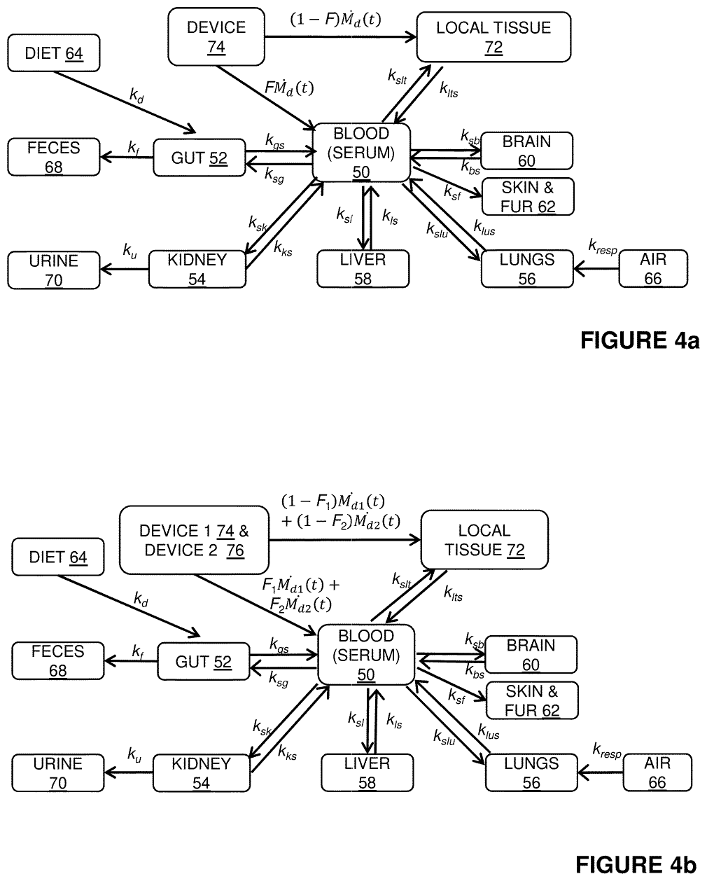

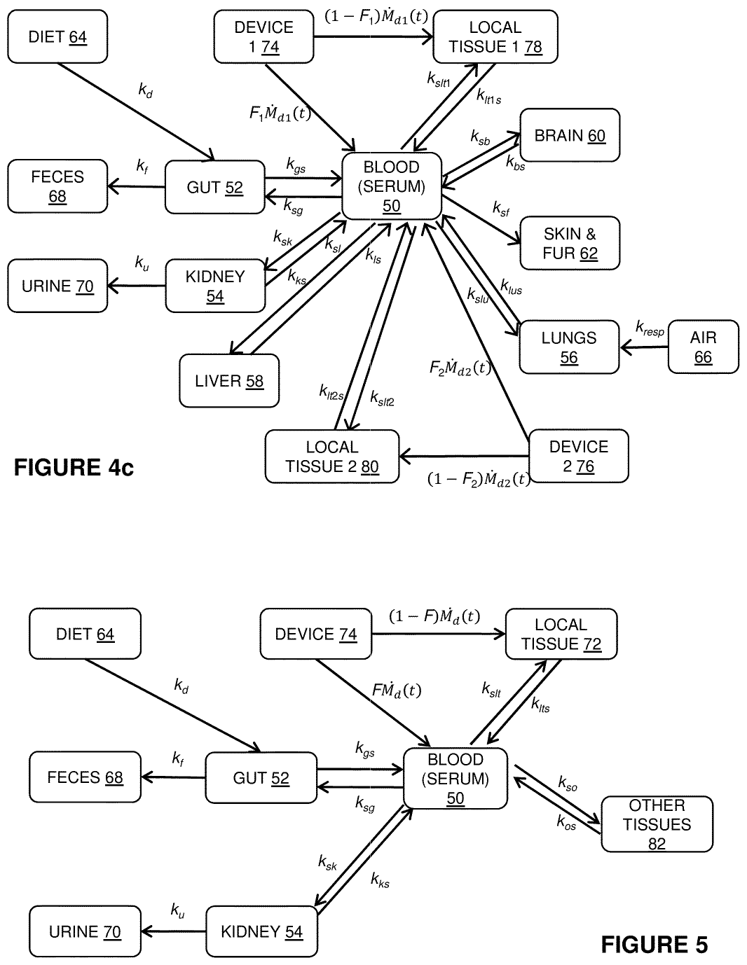

FIG. 4a is a diagram illustrating the diffusion, absorption, distribution, and excretion principles of the PBTK model for one implanted device. The development of the PBTK model of this invention is based on the compartmental biokinetic model of Sunderman et al. (Sunderman, F. W. et al. Nickel absorption and kinetics in human volunteers. Proc. Soc. Exp. Biol. Med. 191, 5-11 (1989)) and the work of Saylor et al.(Regul. Toxicol. Pharmacol. 80, 1-8 (2016)). We signalize that the distant tissue represented cumulatively by Saylor et al. is actually comprised of various organs and thus we further analyze the exchange of a substance (ions and/or particles) between blood (for instance, serum) 50 and the following organs: gut 52, kidney 54, lungs 56, liver 58, brain 60 and skin-fur/hair 62. Under normal dietary ingestion and environmental exposure to the specific substance of interest, the model of this invention (depicted in FIG. 4a) is comprised of a zero-order rate of dietary (k_{d}) absorption (from diet 64) and of inhalation (Kᵣₑₛₚ) absorption (from air 66), a first-order elimination from the gut 52 to feces 68 (k_{f}), from the kidney 54 to urine 70 (kᵤ), and from the blood (serum) 50 to skin-fur/hair 62 (K_{skf}), and a first-order exchange between blood (serum) 50 and local tissue 72 (kₗₜₛ and Kₛₗₜ), liver 58 (Kₗₛ and Kₛₗ), brain 60 (K_{bs} and K_{sb}), lungs 56 (Kₗᵤₛ and Kₛₗᵤ), kidneys 54 (Kₖₛ and Kₛₖ), and gut 52 (K_{gs} and k_{sg}).

To account for the released substance from the implanted device we follow Saylor et al. and introduce two further compartments in the model of this invention: the device 74 and the local tissue 72 adjacent to the device. As a source, the medical implant device 74 generates the substance of interest at a rate of \dot{M}_{d}=dM_{d}/dt, where M_{d} is the mass of substance released from the device and t is time. The device 74 is expected to release ions and/or particles at a rate of which a fraction F is released directly into the blood (in particular the serum) 50 and the rest into the local tissue 72.

The equations to be solved then are:

$${\dot{M}}_{lt} ( t ) = ( 1 - F ( t ) ) {\dot{M}}_{d} ( t ) - {k}_{lts} ( t ) {M}_{lt} ( t ) + {k}_{slt} ( t ) {M}_{s} ( t ) , \qquad ( E1 )$$

$${\dot{M}}_{s} ( t ) = F ( t ) {\dot{M}}_{d} ( t ) + {k}_{lts} ( t ) {M}_{lt} ( t ) - ( {k}_{s l} ( t ) + {k}_{s k} ( t ) + {k}_{s l u} ( t ) + {k}_{s g} ( t ) + {k}_{s b} ( t ) + {k}_{slt} ( t ) + {k}_{s f} ( t ) ) {M}_{s} ( t ) + {k}_{l s} ( t ) {M}_{l i v} ( t ) + {k}_{k s} ( t ) {M}_{k} ( t ) + {k}_{l u s} ( t ) {M}_{l u n g} ( t ) + {k}_{g s} ( t ) {M}_{g} ( t ) + {k}_{b s} ( t ) {M}_{b} ( t ) ,$$

$${\dot{M}}_{k} ( t ) = - ( {k}_{k s} ( t ) + {k}_{u} ( t ) ) {M}_{k} ( t ) + {k}_{s k} ( t ) {M}_{s} ( t ) ,$$

$${\dot{M}}_{l i v} ( t ) = - {k}_{l s} ( t ) {M}_{l i v} ( t ) + {k}_{s l} ( t ) {M}_{s} ( t ) ,$$

$${\dot{M}}_{l u n g} ( t ) = - {k}_{l u s} ( t ) {M}_{l u n g} ( t ) + {k}_{s l u} ( t ) {M}_{s} ( t ) + {k}_{r e s p} ( t ) ,$$

$${\dot{M}}_{g} ( t ) = - ( {k}_{g s} ( t ) + {k}_{f} ( t ) ) {M}_{g} ( t ) + {k}_{s g} ( t ) {M}_{s} ( t ) + {k}_{d} ( t ) ,$$

$${\dot{M}}_{b} ( t ) = - {k}_{b s} ( t ) {M}_{b} ( t ) + {k}_{s b} ( t ) {M}_{s} ( t ) ,$$

$${\dot{M}}_{u} ( t ) = {C}_{u} ( t ) {Q}_{u} = {k}_{u} ( t ) {M}_{k} ( t ) ,$$

$${\dot{M}}_{f} ( t ) = {C}_{f} ( t ) {Q}_{f} = {k}_{f} ( t ) {M}_{g} ( t ) ,$$

$${\dot{M}}_{s f} ( t ) = {C}_{s f} ( t ) {Q}_{s f} = {k}_{s f} ( t ) {M}_{s} ( t ) ,$$

Here, Mₗₜ, Mₛ, Mₖ, Mₗᵢᵥ, M_{lung}, M_{g}, Mᵤ, M_{f}, M_{sf}, and M_{b} denote the mass of the substance in the local tissue 72 (adjacent to the device 74), blood (serum) 50, kidney 54, liver 58, lungs 56, gut 52, urine 70, feces 68, skin/fur 62, and brain 60 compartments, respectively. Also, Cᵤ, C_{f}, and C_{sf}, are the substance concentrations in the urine 70, feces 68, and skin/fur 62, respectively, and Qᵤ, Q_{f}, and Q_{sf}, the volumetric urine 70, feces 68, and sweat outputs. The kinetic parameters kᵢ can be either constants or time-dependent functions (e.g., a polynomial, a Gaussian pulse, a mix of exponentials, etc.), whose exact functional form needs not be specified here, depending on the data to be fitted.

The cumulative amount of substance released from the device, M_{d}(t), and thus the release rate of the substance from the device, \dot{M}_{d}(t), can be obtained either using an analytical expression, or via the use of a multi-scale (material, tissue, and system) biokinetic model wherein the release of the substance is dictated from a diffusion (partial differential) equation. Alternatively, the release rate from the device may be obtained by considering the local diffusion through the tissue and its interaction with the device itself.

For cardiovascular implants (e.g. stents, heart valves, atrial occluders), more elaborate mathematical equations under certain conditions may be used to describe the mass transfer between the device and blood and the peri-implant tissue via the use of mass transfer coefficients. These equations would include the fluid flow equations (e.g., continuity, momentum, and energy equations) and the mass balance of the substance. In this case, the appropriate initial and boundary conditions would need to be employed. These boundary conditions may be either constant or a function of time and/or space, and the boundary conditions may be different at the inflow/outflow surfaces (at a certain distance downstream and upstream of the device). Also, these initial and boundary conditions could be determined either via the use of empirical equations, clinical data, mathematical formulae, or a combination thereof. Blood may be represented rheologically as a Newtonian or a non-Newtonian fluid, and constant or pulsatile blood flow may be used. The peri-implant tissue (arterial or endocardium) may be rigid or flexible or a combination thereof. The mathematical equations and the initial and boundary conditions can be solved using conventional mathematical or numerical techniques, which include analytical or special functions, numerical methods (such as finite differences, finite elements, spectral methods, finite volumes, etc.), methods that could use machine-learning, or a hybrid method that combines two or more of the aforementioned methods.

The method and system described herein provides the predicted toxicokinetic profiles of the interested substance in a mammalian tissue or biofluid. These output data from the invention may be utilized by medical implant manufacturers, implant R&D companies, the medical device testing industry or any other entity to optimize their iterative design process and minimize their development costs. Research Institutes and Universities doing research on novel designs of medical implants may also use the prognostic tool to facilitate enhancements in material properties and design characteristics. The increasing need of verification and validation for medical devices and imposition of stringent government regulations to ensure that the products comply with the quality, safety, and performance standards have led regulatory bodies worldwide to strongly recommend the use of modeling and simulation tools to support medical device submissions. The consistency and predictability of in silico models will eventually enable their use by physicians to improve decision-making in clinical practice. FIG. 4b is a diagram illustrating the diffusion, absorption, distribution, and excretion principles of the PBTK model with two devices at a single implantation site.

The model can be applied when there are multiple devices (same or different type) at different sites or region of implantation or when the single device or various parts comprising a device (e.g., orthotopic implants) degrade at different time rates. All the aforementioned examples, can be considered via the use of a particular time-dependent expression for the release rate, as obtained from appropriate in vitro testing setups simulating the specific in vivo environment of each particular device. For example, consider that at time t=0 the first device 74 is implanted, whereas the second device 76 is implanted at time t=t₀; then the release rate of the substance from the second device \dot{M}_{d2}(t) would be null for all t<t₀. The precise time-dependency of these release rates, \dot{M}_{dk} (t), k=1, 2 . . . n, is obtained from in vitro experiments for each device.

For the case of two devices, the equations to be solved then are:

$${\dot{M}}_{lt} ( t ) = ( 1 - {F}_{1} ( t ) ) {\dot{M}}_{d 1} ( t ) + ( 1 - {F}_{2} ( t ) ) {\dot{M}}_{d 2} ( t ) - {k}_{lts} ( t ) {M}_{lt} ( t ) + {k}_{slt} ( t ) {M}_{s} ( t ) , \qquad ( E2 )$$

$${\dot{M}}_{s} ( t ) = {F}_{1} ( t ) {\dot{M}}_{d 1} ( t ) + {F}_{2} ( t ) {\dot{M}}_{d 2} ( t ) + {k}_{lts} ( t ) {M}_{lt} ( t ) - ( {k}_{s l} ( t ) + {k}_{s k} ( t ) + {k}_{s l u} ( t ) + {k}_{s g} ( t ) + {k}_{s b} ( t ) + {k}_{slt} ( t ) + {k}_{s f} ( t ) ) {M}_{s} ( t ) + {k}_{l s} ( t ) {M}_{liv} ( t ) + {k}_{k s} ( t ) {M}_{k} ( t ) + {k}_{l u s} ( t ) {M}_{lung} ( t ) + {k}_{g s} ( t ) {M}_{g} ( t ) + {k}_{b s} ( t ) {M}_{b} ( t ) ,$$

$${\dot{M}}_{k} ( t ) = - ( {k}_{k s} ( t ) + {k}_{u} ( t ) ) {M}_{k} ( t ) + {k}_{s k} ( t ) {M}_{s} ( t ) ,$$

$${\dot{M}}_{l i v} ( t ) = - {k}_{l s} ( t ) {M}_{l i v} ( t ) + {k}_{s l} ( t ) {M}_{s} ( t ) ,$$

$${\dot{M}}_{l u n g} ( t ) = - {k}_{l u s} ( t ) {M}_{l u n g} ( t ) + {k}_{s l u} ( t ) {M}_{s} ( t ) + {k}_{r e s p} ( t ) ,$$

$${\dot{M}}_{g} ( t ) = - ( {k}_{g s} ( t ) + {k}_{f} ( t ) ) {M}_{g} ( t ) + {k}_{s g} ( t ) {M}_{s} ( t ) + {k}_{d} ( t ) ,$$

$${\dot{M}}_{b} ( t ) = - {k}_{b s} ( t ) {M}_{b} ( t ) + {k}_{s b} ( t ) {M}_{s} ( t ) ,$$

$${\dot{M}}_{u} ( t ) = {C}_{u} ( t ) {Q}_{u} = {k}_{u} ( t ) {M}_{k} ( t ) ,$$

$${\dot{M}}_{f} ( t ) = {C}_{f} ( t ) {Q}_{f} = {k}_{f} ( t ) {M}_{g} ( t ) ,$$

$${\dot{M}}_{s f} ( t ) = {C}_{s f} ( t ) {Q}_{s f} = {k}_{s f} ( t ) {M}_{s} ( t ) ,$$

Here, \dot{M}_{d1}(t) is the release rate of the substance from the first device, and \dot{M}_{d2}(t) the release rate of the substance from the second device, and F₁ and F₂ are the fraction of the substance released directly into the blood and the local tissue from the first and the second medical implant device, respectively. All other parameters are defined as noted above with respect to equations E1.

In this case, the qualitative concentration profile, will, in general, feature two distinct peaks as illustrated in provided in FIGS. 1e and 1f. Note that in the case shown in FIGS. 1e and 1f, as an example, the second peak exceeds the toxicological threshold.

FIG. 4c illustrates diagrammatically the PBTK model with two devices implanted at different sites or regions, wherein the first device 74 is implanted in the vicinity of first local tissue 78, and the second device 76 is implanted it the vicinity of second local tissue 80 (where first 78 and second 80 local tissues are at different sites).

The equations to be solved then are:

$${\dot{M}}_{lt 1} ( t ) = ( 1 - {F}_{1} ( t ) ) {\dot{M}}_{d 1} ( t ) - {k}_{lt 1 s} ( t ) {M}_{lt 1} ( t ) + {k}_{slt 1} ( t ) {M}_{s} ( t ) , \qquad ( E3 )$$

$${\dot{M}}_{lt 2} ( t ) = ( 1 - {F}_{2} ( t ) ) {\dot{M}}_{d 2} ( t ) - {k}_{lt 2 s} ( t ) {M}_{lt 2} ( t ) + {k}_{slt 2} ( t ) {M}_{s} ( t ) ,$$

$${\dot{M}}_{s} ( t ) = {F}_{1} ( t ) {\dot{M}}_{d 1} ( t ) + {F}_{2} ( t ) {\dot{M}}_{d 2} ( t ) + {k}_{lt 1 s} ( t ) {M}_{lt 1} ( t ) + {k}_{lt 2 s} ( t ) {M}_{lt 2} ( t ) - ( {k}_{s l} ( t ) + {k}_{s k} ( t ) + {k}_{slu} ( t ) + {k}_{s g} ( t ) + {k}_{s b} ( t ) + {k}_{slt 1} ( t ) + {k}_{slt 2} ( t ) + {k}_{s f} ( t ) ) {M}_{s} ( t ) + {k}_{ls} ( t ) {M}_{liv} ( t ) + {k}_{k s} ( t ) {M}_{k} ( t ) + {k}_{lus} ( t ) {M}_{lung} ( t ) + {k}_{g s} ( t ) {M}_{g} ( t ) + {k}_{b s} ( t ) {M}_{b} ( t ) ,$$

$${\dot{M}}_{k} ( t ) = - ( {k}_{k s} ( t ) + {k}_{u} ( t ) ) {M}_{k} ( t ) + {k}_{s k} ( t ) {M}_{s} ( t ) ,$$

$${\dot{M}}_{liv} ( t ) = - {k}_{ls} ( t ) {M}_{liv} ( t ) + {k}_{s l} ( t ) {M}_{s} ( t ) ,$$

$${\dot{M}}_{lung} ( t ) = - {k}_{lus} ( t ) {M}_{lung} ( t ) + {k}_{slu} ( t ) {M}_{s} ( t ) + {k}_{r e s p} ( t ) ,$$

$${\dot{M}}_{g} ( t ) = - ( {k}_{g s} ( t ) + {k}_{f} ( t ) ) {M}_{g} ( t ) + {k}_{s g} ( t ) {M}_{s} ( t ) + {k}_{d} ( t ) ,$$

$${\dot{M}}_{b} ( t ) = - {k}_{b s} ( t ) {M}_{b} ( t ) + {k}_{s b} ( t ) {M}_{s} ( t ) ,$$

$${\dot{M}}_{u} ( t ) = {C}_{u} ( t ) {Q}_{u} = {k}_{u} ( t ) {M}_{k} ( t ) ,$$

$${\dot{M}}_{f} ( t ) = {C}_{f} ( t ) {Q}_{f} = {k}_{f} ( t ) {M}_{g} ( t ) ,$$

$${\dot{M}}_{s f} ( t ) = {C}_{s f} ( t ) {Q}_{s f} = {k}_{s f} ( t ) {M}_{s} ( t ) ,$$

Here, Mₗₜ₁(t) and Mₗₜ₂(t) is the mass of the substance in the first and the second local tissue, respectively. Kₗₜ₁ₛ and kₗₜ₂ₛ are the rates of exchange from the first local tissue to blood (serum) and second local tissue to blood (serum), respectively. Kₛₗₜ₁ and Kₛₗₜ₂ are the rates of exchange from blood (serum) to the first local tissue, and from blood (serum) to the second local tissue, respectively. All other parameters are defined as noted above with respect to equations E1 and E2.

FIG. 5 is a diagram illustrating the diffusion, absorption, distribution, and excretion principles of a coarse-grained or more generalized version of the PBTK model with a single implanted device.

The execution of in vivo experiments is a laborious and a costly procedure. As such, in many studies only a limited number of samples are analyzed, with primary emphasis given to obtaining samples from blood and or excrements. In other cases, tissues or biofluids that are pharmacokinetically and toxicologically similar can be lumped/grouped together (J. L. Campbell Jr., R. A. Clewell, P. R. Gentry, M. E. Andersen, and H J. Clewell III, “Physiologically Based Pharmacokinetic/Toxicokinetic Modeling”, Ch 18 in Computational Toxicology: Volume I, B. Reisfeld and A. N. Mayeno (eds.), Methods in Molecular Biology, vol. 929 (2012)). In such an approach, the model development starts on the version that has the finer information, and decisions are made to coarse grain some elements (tissues and biofluids) together, provided this can be justified.

One possible grouping could be undertaken when the respiratory intake rate is negligible, and grouping the brain, skin/fur, the lungs, and the liver into a new compartment named “other tissues” 82 (see FIG. 5). Then, we may define two new kinetic parameters as kₛₒ(t)=k_{sb}(t)+kₛₗᵤ(t)+Kₛₗ(t)+k_{skf}(t) and

$${k}_{o s} ( t ) = \frac{{M}_{s}}{{M}_{o}} {k}_{b s} ( t ) + \frac{{M}_{lung}}{{M}_{o}} {k}_{l u s} ( t ) + \frac{{M}_{liv}}{{M}_{o}} {k}_{l s} ( t )$$

where Mₒ is the mass of the substance in the “other tissues”, so that the equations to be solved in this case are:

$${\dot{M}}_{lt} ( t ) = ( 1 - F ( t ) ) {\dot{M}}_{d} ( t ) - {k}_{lts} ( t ) {M}_{lt} ( t ) + {k}_{slt} ( t ) {M}_{s} ( t ) , \qquad ( E4 )$$

$${\dot{M}}_{s} ( t ) = F ( t ) {\dot{M}}_{d} ( t ) + {k}_{lts} ( t ) {M}_{lt} ( t ) - ( {k}_{s k} ( t ) + {k}_{s o} ( t ) + {k}_{s g} ( t ) + {k}_{slt} ( t ) ) {M}_{s} ( t ) + {k}_{o s} ( t ) {M}_{o} ( t ) + {k}_{k s} ( t ) {M}_{k} ( t ) + {k}_{g s} ( t ) {M}_{g} ( t ) ,$$

$${\dot{M}}_{k} ( t ) = - ( {k}_{k s} ( t ) + {k}_{u} ( t ) ) {M}_{k} ( t ) + {k}_{s k} ( t ) {M}_{s} ( t ) ,$$

$${\dot{M}}_{o} ( t ) = - {k}_{o s} ( t ) {M}_{o} ( t ) + {k}_{s o} ( t ) {M}_{s} ( t ) ,$$

$${\dot{M}}_{g} ( t ) = - ( {k}_{g s} ( t ) + {k}_{f} ( t ) ) {M}_{g} ( t ) + {k}_{s g} ( t ) {M}_{s} ( t ) + {k}_{d} ( t ) ,$$

$${\dot{M}}_{u} ( t ) = {C}_{u} ( t ) {Q}_{u} = {k}_{u} ( t ) {M}_{k} ( t ) ,$$

$${\dot{M}}_{f} ( t ) = {C}_{f} ( t ) {Q}_{f} = {k}_{f} ( t ) {M}_{g} ( t ) ,$$

Note that if the average percentage of the substance(s) in the tissues that are grouped is, approximately, constant and known, then the grouping can take place directly. If the average percentages are not constant, then the training mode should again be initiated.

Another consideration is the nature of the substances. Substances can be considered predominantly as ions, while it is also possible to have non-ionic particles at micro and/or nano scale. In general, the electrochemical processes that drive biodegradation and corrosion lead to both dissolution of materials (as ions) as well as surface deposition (corrosion products e.g., polymeric nano- or microparticles released from a device e.g., drug-eluting stents). Based on the solubility of the dissolved ions, it is possible for the ions to precipitate out to form additional corrosion products. The release may be simultaneous or with a time lag. The surface deposition products are localized to areas near the corrosion location, corrosion precipitates are generally found on the device or in adjacent tissue, while the dissolved ions can be found systemically (i.e., around the wider system). Particles and ions diffuse differently; the diffusion of a particle in blood depends mainly on its size and shape, blood viscosity, steric interactions, and buoyancy; also, particles are known to migrate towards the vascular wall. On the other hand, the diffusion of ions is mainly dictated by electrostatic interactions (e.g., by using the Debye-Hűckel equation), but also on its size and blood viscosity.

The spread of corrosion products throughout the body makes definitive biodegradation product studies a challenge. Most rely on analysing the surface deposited/precipitated products while others include local tissue response. While many molecular, cellular, and signalling pathways associated with inflammatory and immune responses to implants are shared across device types, intended uses, anatomy, and physiology, there is a growing appreciation for device-tissue interface as an independent dimension of functional specialization in inflammatory and immune processes. As described in the CDRH Biocompatibility Guidance, for biocompatibility tests requiring extraction of the device, single or multiple extracts may be needed to assess both polar and non-polar chemicals that can be released from the device.

Therefore, the various model equations and parameters are tuned accordingly to enable predictions for single or multiple types and forms of substances.

In order to check the accuracy of the proposed model, the concentration of ions in all samples may be measured using high-resolution inductively coupled plasma mass spectrometry (ICP-MS). ICP-MS is the most widely used method today for the determination of concentrations in both biological and inorganic samples. The instrument vaporizes a sample into ions then injects them into a mass spectrometer. The mass spectrometer separates and detects the ions according to their mass to charge ratio, and measures the analyte concentration by mass fractionation, providing a very sensitive quantitative method for analyte detection. ICP-MS's strengths include: simultaneous detection of many elements or ions; a low detection limit; and wide linear calibration range. Inductively coupled plasma optical emission spectroscopy (ICP-OES) and atomic absorption spectroscopy (AAS) can also be used. Differences between instrumentation and sample preparation prior to testing could complicate the analysis.

Tissue, biofluid and excrement samples are collected from animals implanted with a medical device, for ion analysis, but also from healthy animals to establish baseline concentrations of the specific ion. Ion levels are also measured in animal food and water to calculate the average daily dietary uptake. Tissue surrounding explanted devices is removed by digestion in NaOH solution based on previous results demonstrating it does not significantly alter implant surfaces (Sullivan S J L, Stafford P, Malkin E, Dreher M L, Nagaraja S. 2018. Effects of tissue digestion solutions on surface properties of nitinol stents. J Biomed Mater Res Part B 2018:106B:331-339). Samples are placed in 1 M solution of NaOH (NaOH volume to specimen surface area ratio ˜0.05 ml/mm²) and incubated at 37° C. for 48 hours to dissolve away the tissue. The tissue digested solution is then collected for ICP-MS analysis. All biological specimens are digested using a closed-vessel microwave digestion procedure, which is appropriate to the matrix of the sample, before analysis via ICP-MS. In order to monitor metallic contamination throughout all steps, ion levels are also measured in appropriate control samples and all tools and containers used during the tissue removal process and the handling of the explants are non-metallic and acid-washed using a 10% HNO₃ solution prior to use. All samples are acidified with 2% HNO₃ before analysis to ensure stability and comparability with calibration standards. Raw measurements are obtained in units of ppb (μg/kg).

To assess general corrosion and ion release, in vitro immersion testing is often conducted to quantify ion release over time under physiologically relevant conditions as per ASTM F3306 “Standard Test Method for Ion Release Evaluation of Medical Implants.”. This testing typically consists of placing the device in a container filled with media representative of the implant environment and storing it for a predetermined duration. At subsequent time intervals, the media is sampled for chemical analysis using methods such as inductively coupled plasma mass spectrometry (ICP-MS). This testing is usually conducted for a sufficient duration to establish that ion release has reached steady-state or approached equilibrium, and the time intervals are selected to adequately capture the extent of any initial bolus release from the device. In addition to providing a framework for comparing different alloys, designs, or manufacturing processes, immersion test data are used to estimate exposure as part of toxicological risk assessment (e.g., per ISO 10993-17). The American Standard of Testing and Materials (ASTM) recently published ASTM F3306 “Standard Test Method for Ion Release Evaluation of Medical Implants” that describes test methods to assess ion release from medical implants (ASTM 2019).

In vitro test methods can provide significant insight into the corrosion susceptibility of a given device. However, they are typically conducted under idealized and/or hyper-physiological conditions. Thus, while these tests enable comparisons between devices to be readily made, the extent to which in vitro performance correlates to corrosion behavior in vivo remains unclear. This is primarily due to the scarcity of relevant in vivo data. Significantly different corrosion behaviors have been documented in non-physiological in vitro tests, physiological in vitro tests, and in vivo studies. Although qualitative consistency between engineering testing and behavior inside the body between devices exists, quantification of these relationships using computer models, engineering testing, tissue, and ex vivo body fluid evaluations, testing within the human body (e.g., imaging), and/or clinical studies is important for corrosion prediction within individual patients. Therefore, uncertainty in patient exposure is one of the largest knowledge gaps to establishing patient risk associated with corrosion products generated by implantable devices.

Although linking the results of in vitro testing to in vivo outcomes presents formidable challenges, available data do suggest there are at least qualitative consistencies. Modeling and simulation tools represent a promising and relatively easy way to potentially establish these relationships quantitatively. Toxicokinetic models can link in vivo ion release to easily measured clinical parameters, such as blood (serum) or urine ion concentrations, enabling in vivo exposure inferred from these measurements to be compared directly to the results of in vitro testing. Establishing in vitro to in vivo correlations requires a combination of computational modeling and in vitro, ex vivo, and in vivo testing as well as clinical studies to inform and validate the model predictions.

Since an FDA guidance document (Technical Considerations for NonClinical Assessment of Medical Devices Containing Nitinol Guidance for Industry and Food and Drug Administration Staff. Document issued on Jul. 9, 2021) recommends that testing should be performed on as-manufactured devices after subjecting the device to simulated-use conditions, the technology of this invention employs appropriate testing setups (experimental, theoretical and computational modeling) that allow for in vitro release measurements representative of the in situ biomechanochemical loading. This type of testing should closely mimic physiologic conditions and capture both the initial bolus release of the substance as well as the longer-term release profile in vitro. This test setup can be readily modified and customized for different types of medical devices and the mechanical and biochemical environment at the specific site of implantation for which the substance release profile combined with mechanical loading is desired.

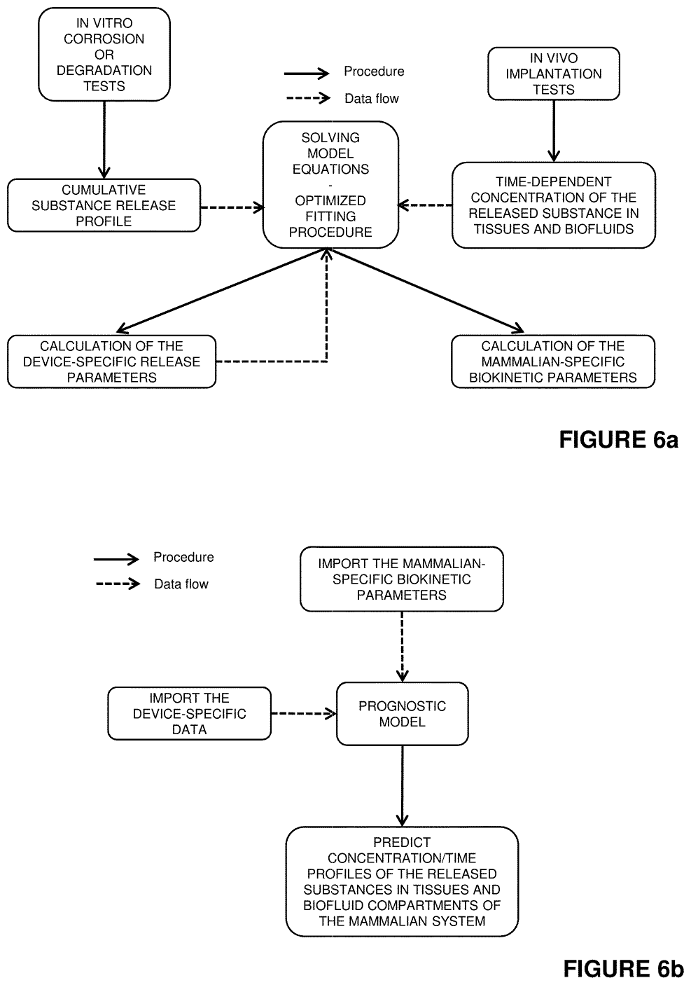

FIGS. 6a and 6b are diagrammatic illustrations of the process of estimating model parameters and the model-derived prognosis for a specific mammalian type according to the claimed method. According to FIG. 6a, the model (comprising of model equations E1, E2, E3 or E4) is solved after taking inputs for certain parameters that have been estimated from in-vitro corrosion and/or degradation studies (that refer to the implantation device) and in-vivo implantation studies in a mammal. The model subsequently can be used to calculate device-specific release parameters and mammalian-specific biokinetic parameters. This is a training phase where model parameters are estimated, as represented in FIG. 6a.

After training the model enters a prognostic phase, as represented by FIG. 6b. The prognostic model (comprising of model equations E1, E2, E3 or E4) makes use of (or imports) the device-specific release parameters and the mammalian specific biokinetic parameters obtained in the training phase. Subsequently, the model can be used to predict concentration-time profiles of the released substance (ions and/or particles) in tissue and biofluid compartments of the mammalian system of interest (such as humans), based on a set of device design parameters (input as device-specific data).

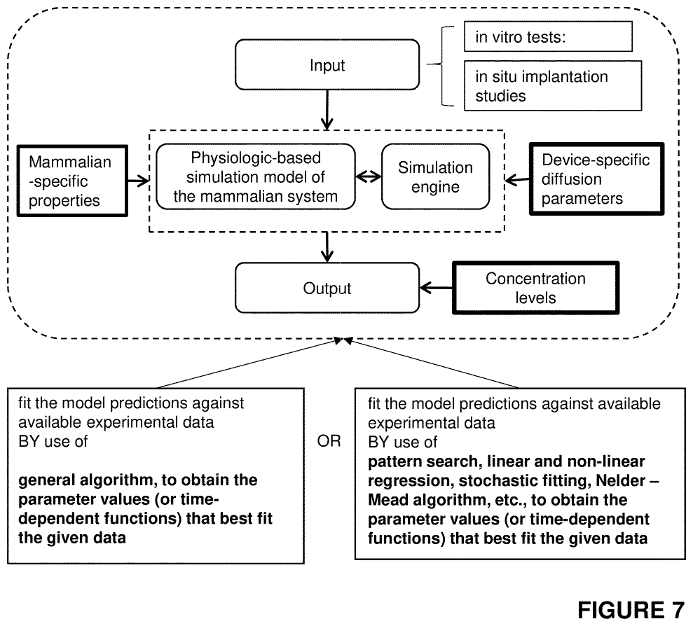

FIG. 7 is a diagrammatic representation of the present method showing alternative means of fitting the model predictions against available experimental data, for use in the training stage. Several approaches to data fitting may be used. For example, algorithmic approaches may be used. Alternatively, a range of numeric methods may be used including but not limited to pattern search, linear and non-linear regression, stochastic fitting, Nelder-Mead algorithm, etc., to obtain the parameter values (or time-dependent functions) that best fit the given data.

As described above, the training stage (FIG. 6a) and predictive stage (FIG. 6b) may be part of a machine-learning system for predicting the toxicokinetic profiles of the interested substance in mammalian tissue or biofluid may include a training mode and a production mode. During the training mode (FIG. 6a), appropriate in vitro testing is performed (experimental, theoretical and computational modeling) that allows for in vitro release measurements during in situ biomechanochemical loading. This type of testing should closely mimic physiologic conditions and capture both the initial bolus release of the substance as well as the longer-term release profile in vitro. This information is used to obtain the substance release rate, \dot{M}_{d}(t), for each individual case (for instance, a particular implant device design). The remaining kinetic parameters are to be specified through in situ implantation data in an appropriate mammalian model. If needed, these kinetic parameters can be obtained by fitting the model predictions against available in vivo data from a different animal model and then modified using extrapolation algorithms to reflect human or other physiology; all algorithms can be programmed to decide which model parameters, if any, need to be time dependent. The fitting process can include a standard non-linear curve-fitting algorithm, to obtain the best values (or time-dependent functions) of the unknown biokinetic parameters. In a different embodiment, the fitting algorithm may employ a different strategy, such as pattern search, linear and non-linear regression, stochastic fitting, Nelder-Mead algorithm, etc., or any other such approach that may be used to obtain the parameter values (or time-dependent functions) that best fit the given data. This step could also include fitting the model predictions against only a fraction of the available experimental data, and then checking the accuracy of the parameter estimation through the fitting process by predicting the remaining in vivo experimental data.

Subsequently, during the predictive stage, the invention may use the mammalian-specific biokinetic parameters and device-specific release parameters obtained in the training stage to predict the concentration-time profile of said substance in a tissue or organ, a biofluid or an excreta compartment of the mammalian system when one uses devices with different design and/or material characteristics. The model can be applied with a set of device-specific data (representing device design parameters), to predict the particular concentration-time profile of said substance in tissue or biofluid compartments of the mammalian system when one uses a medical implant device with the given design parameters.

Given the full parameterization of the model during the training mode, its outcomes can be used in the predictive stage (FIG. 6b). During this stage, the invention may use the mammalian-specific biokinetic parameters obtained in the training mode and device-specific release parameters obtained by in vitro testing or simulation to predict the concentration-time profile of said substance in tissue or biofluid compartments of the said mammalian system when one uses devices with different design and/or material characteristics or site of implantation. The model can be applied with a set of device-specific data (representing device design parameters and implantation site parameters), to predict the particular concentration-time profile of said substance in tissue or biofluid compartments of the mammalian system when one uses a medical implant device with the given design parameters and the given implantation site. Consequently, a predicted peak in the concentration-time profile of a substance in a given tissues and biofluid can be compared to a predefined exposure threshold (or toxicological threshold) to identify possible violations of the threshold.

In further examples, a perturbation or adjustment to the implantation site and the design and/or material characteristics of the device can be applied within the model (in other words, a perturbation or adjustment to the device-specific data). A comparison of the predicted peak in the concentration-time profile of a substance in a given tissues and biofluid for the new, adjusted device design can be compared to a predefined exposure threshold (or toxicological threshold). In this way, an optimum solution, i.e., a medical implant device design and implantation site that results to the least exposure to toxicologically hazardous levels of said substance, can be found.

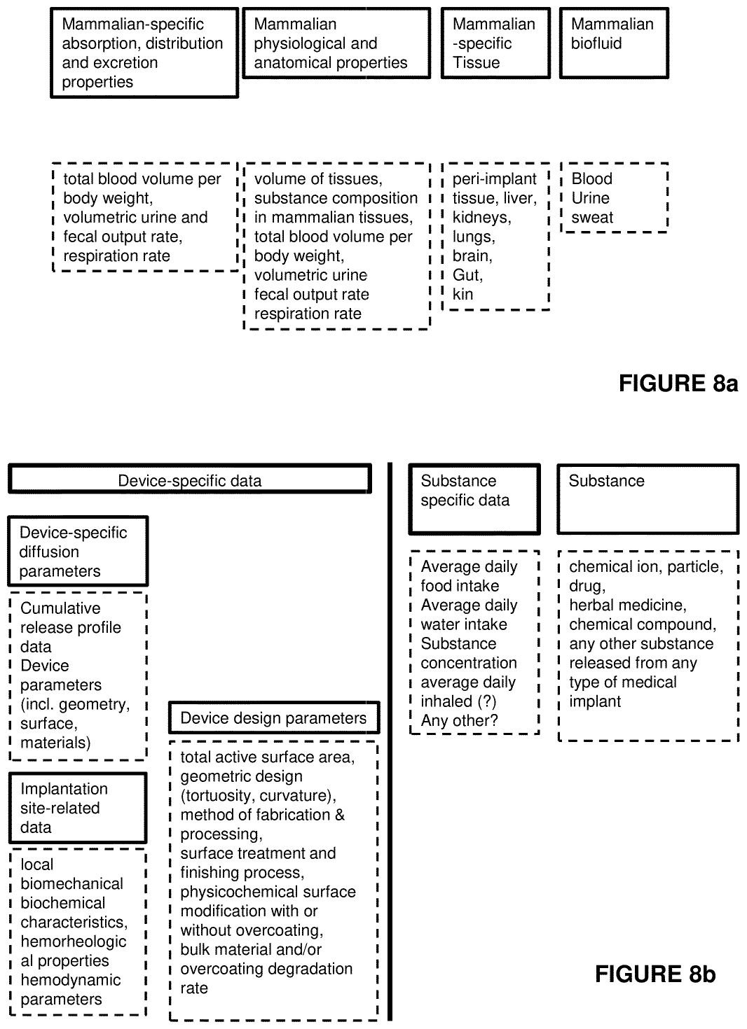

Several mammalian-specific parameters may be included in the model. Mammalian-specific parameters are parameters specific to the particular mammal into which the medical device will be implanted. FIG. 8a outlines mammalian-specific parameters organised in four groups: mammalian-specific absorption, distribution and excretion properties; mammalian physiological and anatomical properties; mammalian-specific tissue; and, mammalian biofluid.

Mammalian physiological and anatomical properties of mammalian-related data include gender, weight, height, heart and breath rate. In addition, mammalian physiological parameters also include exercise, lifestyle, and age. Mammalian-specific data may also comprise socio-biographical data age, race and ethnicity groups, lifestyle, eating and smoking habits, environmental and exposure conditions at the place of residence and work.

Biomonitoring measures the internal dose of a chemical substance resulting from integrated exposures from all exposure routes. It has been used increasingly as a tool for quantifying human exposure to chemicals for risk assessment and risk management decisions. Model parameterization includes the mammalian-specific baseline concentrations and intake of the specific chemical substance for healthy individuals. The reference population can include different age, sex, race and ethnicity groups, lifestyle, eating and smoking habits, environmental and exposure conditions at the place of residence and work.

Similarly, several device-specific and substance-specific data may be included in the model. FIG. 8b outlines device-specific data (including device design parameters, device-specific diffusion parameters, and implantation site-related data) and substance-specific data. Device design parameters may comprise total active surface area, geometric descriptors, characterization of method of fabrication in relation to quantified release of substances, and surface treatment in relation to quantified release of substances. These are some of device design parameters, which a manufacturer may amend as part of the optimization process in order to reduce the toxicological risk of the medical implant device. Some of these parameters, such as total surface area, may have a direct value, while other device design parameters, such as geometric complexity or surface treatment, may be represented through appropriate characterizations that are suitable for modelling purposes.

Local biomechanical and biochemical characteristics may also be included within the model, and may include: dynamic geometries, cyclic and dynamic loading profiles, and chemical conditions (chloride, dissolved oxygen, and PH levels). For intravascular devices, other implantation site-related data may also be included within the model, and may comprise: implantation site, anatomical characteristics of blood vessel(s), local biomechanical (geometric and loading parameters) and biochemical characteristics, hemorheological properties and hemodynamic parameters that describe the local and peripheral blood flow profile.

The in-silico tool and methods of the invention can also be utilized to interpret ion biomonitoring but also serve as the basis for reverse dosimetry. If a level of release from the device associated with toxicological concern is established, the resulting critical serum (or blood) and urine concentrations can be specified. Through monitoring these levels, it is possible to assess whether the release from a comparable device is likely exceeding levels associated with toxicological concern. Based on prediction, device design can be optimized to ensure that concentrations of released substances will remain under permissible exposure limits. The optimization is done through an iterative process that comprises the perturbation of device-specific parameters.

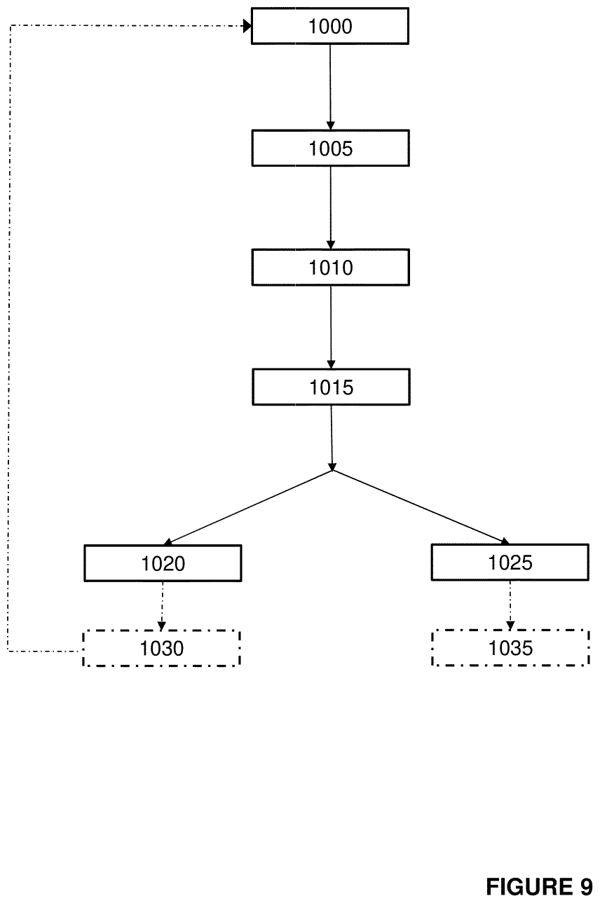

An aspect of the invention is illustrated in FIG. 9. In particular, there is a method for toxicological risk assessment of a medical implant device, the device for implant into the body of a mammal, wherein a substance is released from the medical implant device after implantation, comprising:
a) (step 1000) inputting device-specific data into a physiologically based toxicokinetic (PBTK) model for the distribution of the substance in the body of the mammal into which the medical implant device is to be implanted, wherein the device-specific data characterises the medical implant device and/or its behaviour at a specific site of implantation;
b) (step 1005) determining, from the PBTK model, a time dependent function for the concentration level of the substance in a tissue, organ, biofluid and/or excreta of the mammal;
c) (step 1010) determining, using the time dependent function for the concentration level of the substance, the maximum of the concentration level during a predetermined time interval;
d) (step 1015) comparing the maximum of the concentration level with a predefined exposure threshold for the tissue, organ, biofluid and/or excreta;

wherein if the maximum of the concentration level exceeds or is equal to the predefined exposure threshold, then identifying (step 1020) the medical implant device characterised by the device-specific data as having an unsatisfactory toxicological risk; or if the maximum of the concentration level is less than the predefined exposure threshold then identifying (step 1025) the medical implant device characterised by the device-specific data as having a satisfactory toxicological risk.

Medical implant devices describe devices that are intended to be placed inside the body (in vivo) in order to provide support or functions to bodily organs, to deliver medication, to monitor bodily functions, or to provide bodily enhancements. Although such implants may offer many medical benefits, by their nature of being placed inside the human body, they may also pose a risk. A particular risk is the release of a substance such as a toxins, toxicants or drug from the implant device which, at too high concentrations, may be harmful to the body in which the implant is placed. The substance may be identified within tissues local to the implant, or may be distributed through the body's circulatory system and so be found in tissues and organs in the body that are not local to the implant. The substance may also be seen in the excreta from the body in which the medical device is implanted. The design of medical implant devices (and the amount of substance released from said devices) may be optimized for the general population or for specific patient groups. Optimization of the design of medical implants may be undertaken with a focus on a specific tissue group so that concentration levels are within acceptable safe threshold levels for said specific tissue group.

The present method looks to assess the risk to a body in which a specific medical implant device is implanted. In particular, the method assesses the toxicity risk—in other words whether the concentration of a substance or substances in any tissue in or excreta from the body exceeds a level that is deemed to be safe (for example, as assessed and determined by regulatory bodies). Medical implants can only be used and implanted within a patient if the toxicity risk is deemed or assessed to be satisfactory (and so not to pose a danger to the health of the patient).

The modelling of the distribution and accumulation of a substance (such as a potential toxin or toxicant) released from a medical implant device in different parts of the body is complex. The present inventors have developed a physiologically based toxicokinetic (PBTK) model to describe the release of a substance (or a reactant of a chemical reaction subsequently forming the substance) from a medical implant device, and then the distribution of the substance to different compartments or elements of the mammalian body. Physiological toxicokinetic models (PBTK) are models developed to describe and predict the behaviour of a toxicant in an animal body; for example, quantifying which parts (compartments) of the body the substance may tend to enter (e.g., lungs, liver, gut, etc.), and whether or not the chemical is expected to be metabolized or excreted and at what rate. The substance may be any type or species of ions, molecules or particles of interest (including metals, polymerics or ceramics). The medical implant device may comprise or contain or be formed from (at least in part) the substance, or comprise or contain or be formed from (at least in part) a reactant of a chemical reaction forming the substance (for instance, when released into the body of the mammal into which the medical implant device is implanted). The substance (or reactant) is releasable from the medical implant device.

The described model and method can be used to improve and optimise device design during manufacture, in order to identify a new design for a medical implant device. Alternatively or additionally, the described model and method can be used to undertake a toxicological risk assessment (and consequently optimise the toxicological risk) of known, commercially available medical implant devices in order to identify the best location for an implantation site within a mammal body. The best location may be the location having lowest toxicological risk, or at which the lowest concentration level of a particular substance released from the medical implant is observed.

The specific PBTK model of the present invention is described in more detail above (in particular with reference to sets of equations E1, E2, E3 and E4) and below (with reference to equations E5 and E6). The proposed PBTK model represents a set of differential equations (specifically, rate equations having time as the independent variable). By solving the set of differential equations, a dependent variable can be obtained. In the specific PBTK model proposed by the inventors, for a particular input device-specific data, solving the set of differential equations of the model can obtain a rate of accumulation of the substance in a particular tissue and/or excreta of the mammal as a time-dependent variable (and thereby giving a time dependent function for the concentration level of the substance in the same tissue and/or excreta of the mammal). As such, from the time-dependent variable, the cumulative amount or cumulative concentration of the substance in the particular tissue and/or excreta of the mammal, for a specific device, can be determined.

It will be understood that the cumulative concentration of a substance in a particular organ, tissue, biofluid or excreta is linked to the rate of release of the substance from the medical implant device, both to local tissue and to blood stream. Said rate of release is one of the set of rate equations making up the PBTK model. The rate of release of the substance from the medical implant device may be a steady rate that reflects the gradual degradation of the device. Alternatively, the rate of release from the medical implant device may be a variable rate over time, which reflects a stage of gradual degradation and a stage of rapid degradation.

Device specific data characterises the medical implant device and/or its behaviour at a specific site of implantation. The device specific data may comprise one or more parameters (for instance, a set of parameters), where some or even all of those parameters may be a time dependent function. The device specific data input to the PBTK model may itself be a time-dependent function describing the rate of release of the substance from a specific medical implant device to local tissue (i.e., tissue surrounding or close to the implant) and blood in the blood stream (for instance to serum (i.e. the liquid fraction of clotted blood) or whole blood) when the device is implanted at a particular implantation site within the mammalian body. The rate of release may be a numerical function, for instance a function obtained via in-vitro measurements of a particular device (in other words, measurement in a laboratory, in idealised or example conditions). The in-vitro measurements may take place following experimental, theoretical or computational tests for a given medical implant device. Alternatively, the rate of release may be a function obtained as a simulation. For instance, the rate of release may be a function of one or more device design parameters and/or one or more implantation site-related parameters, being a simulation or model. The device design parameters may be parameters such as the active surface area of the device, a substance characteristic release time, device geometry and/or a substance specific rate constant. Device design parameters may further include the method of manufacture, surface treatment and/or modification, or other characteristics of the material from which the device is made. Implantation site-related data may comprise local biomechanical and/or biochemical characteristics, hemorheological properties and/or hemodynamic parameters that describe the local and peripheral blood flow profile

A maximum magnitude or maximum amplitude (e.g. peak amplitude) of the cumulative amount or cumulative concentration of the substance in the particular tissue and/or excreta of the mammal within a certain time interval can be compared to a threshold. In an alternative, another measure derived from the cumulative amount or cumulative concentration of the substance in the particular tissue and/or excreta of the mammal within a certain time interval can be compared to a threshold. Said other measure could be a time averaged cumulative amount or cumulative concentration, for instance. The threshold may be a predefined exposure threshold determined on the basis of Toxicological Risk Assessment, and may be predefined by a safety or regulation body (such as the FDA). Different threshold concentrations may be applied with reference to different tissue and/or excreta, for different types of mammal, for different substances or differently to the general population compared to specific patient groups. The time interval can be selected to represent the likely duration of implantation of the medical implant device, or the likely lifespan of the patient, for instance.

Optionally, after identifying the medical implant device characterised by the device-specific data as having an unsatisfactory toxicological risk, or after identifying the medical implant device characterised by the device-specific data as having a satisfactory toxicological risk, the method may further comprise outputting the identified toxicological risk to a screen, otherwise communicating the identified toxicological risk to the user, or storing the identified toxicological risk in memory (step 1035). The result is then visible to the user, or can be retrieved later. As such, device-specific data can be saved with an associated toxicological risk assessment.

The PBTK model may predict time-dependent substance release from one implant device. In this example, the PBTK model is described by the set of equations E1 (discussed above), from which for a given device-specific data (Ma, the rate of release of the substance from the one medical implant device) can be obtained a time dependent function for the concentration level of the substance in a tissue and/or excreta of a mammal, wherein one medical implant device is implanted in the body of the mammal.

Alternatively, the PBTK model may predict time-dependent substance release from two implant devices, the two implant devices implanted simultaneously into the same mammal during at least a portion of the period for which the time-dependent substance release is modelled. In an example wherein two separate medical implant devices are implanted in the body of the mammal in a similar bodily location or implantation site, the PBTK model is described by the set of equations E2 (discussed above), from which for a given device-specific data (\dot{M}_{d1}, the rate of release of the substance from a first of the two medical implant devices and \dot{M}_{d2}, the rate of release of the substance from a second of the two medical implant devices) can be obtained a time dependent function for the cumulative concentration level of the substance in a tissue or organ, biofluid and/or excreta of a mammal. In an example wherein two separate medical implant devices are implanted in the body of the mammal in different or spaced apart bodily locations or implantation sites, the PBTK model is described by the set of equations E3 (discussed above), from which for a given device-specific data (\dot{M}_{d1}, the rate of release of the substance from a first of the two medical implant devices and \dot{M}_{d2}, the rate of release of the substance from a second of the two medical implant devices) can be obtained a time dependent function for the cumulative concentration level of the substance in a tissue or organ, biofluid and/or excreta of a mammal. In other words, the PBTK model can be adapted to represent the cumulative concentration of a substance in a mammalian system in which one or more device is implanted.

In addition, the PBTK model may be adapted to represent the concentration of a substance in a mammalian system in which selected organs and/or types of tissue and/or types of excreta may be grouped together to simplify the PBTK model. For instance, this make use of the set of equations E4 (described above) for the PBTK model.

Prior to implementation of steps a to g, the described method may further comprise training the PBTK model. More specifically, the method may comprise training the PBTK model by performing a successive fitting procedure to fit the PBTK model to experimental data, in order to determine values for kinetic parameters within the PBTK model. The experimental data may be obtained from one or more prior in vivo measurements and/or in vitro measurements. The experimental data may be concentration-time profiles for a device with known device specific data (device design parameters and/or implantation site-related parameters), and/or in known mammalian systems (with known parameters). Machine-learning techniques can be used to fit the PBTK model. Machine-learning may use device-specific cumulative release profile data and/or concentration levels of at least one tissue or biofluid compartment of the mammalian system during the training stage. During training, various biokinetic parameters may be obtained, as described above with reference to FIGS. 8a and 8b.

Training the PBTK model may involve inputting mammalian-specific data into the PBTK model, wherein the mammalian-specific data includes characteristics of the mammal into which the medical implant device is to be implanted. Mammalian-specific data may comprise physiological and anatomical properties of mammalian-related data and implantation site-related data. Mammalian-specific data may comprise one or more parameters selected from a group consisting of: total blood volume per body weight, volumetric urine output rate, faecal output rate, respiration rate, age, gender, race, ethnicity, lifestyle, eating habit, smoking habits, environmental exposure conditions (at the place of residence and/or work of the patient), volume and density of selected tissue and organ compartments. In other words, mammalian-specific data may be socio-biographical data, or physiological data.

Training the PBTK model may involve inputting substance-specific data. Substance-specific data may comprise one or more parameters selected from a group consisting of: a concentration of a substance in an average daily food and/or water intake of a typical adult human or a typical subject of the mammalian type, a concentration of the substance inhaled on an average or typical day. The model may consider one substance at a time, or more than one substance. The model may provide predictions (separately or simultaneously) for multiple substances (including ions and or/particles, including metals, polymerics, ceramics).

Training the PBTK model may involve inputting known device-specific data. As noted above, the device-specific data may be a function describing the rate of release of the substance from the device to local tissue and blood in the blood stream (where blood may refer to blood serum or whole blood) at a particular implantation site. The function may be a time dependent function for the rate of release of a substance from a known device, measured in vivo. In this way, device-specific data may comprise the rate of release of a substance from the implant, obtained as a result of previous in vitro corrosion or degradation testing representative of the in situ biomechanochemical loading. Alternatively, the function may be a time dependent function for the rate of release of a substance from a known device based on a set of (one or more) device design parameters and/or one or more implantation site-related parameters. The device design parameters and/or one or more implantation site-related parameters may individually and/or collectively affect the rate of release of the substance from the implant. Said device design parameters may comprise one or more parameters selected from a group comprising: total surface area of the implant device, method of manufacture of the implant device, surface treatment of the implant device, geometry of the implant device, thickness of layers in a layered construction at the surface of the implant device, material from which the device is made (raw material or alloy), total active surface area of the implant device (affecting diffusion flux), geometric descriptors (3D design, sharp edges, stress raisers (sharp edges, grooves etc,) connector links), method of fabrication or manufacture (forming, casting, powder processing, rapid manufacturing, welding, machining), and surface treatment/modification in relation to quantified release of substances (physicochemical (with or without overcoating, patterning) and biological surface modification techniques), substance specific rate constants, a substance characteristic release time. Said implantation site-related data may comprise one or more parameters selected from a group comprising: local biomechanical and/or biochemical characteristics, hemorheological properties and hemodynamic parameters that describe the local and peripheral blood flow profile. The local biomechanical characteristics or environment (motion, force, momentum, levers and balance) at an implantation site can be directly affected by the local geometry of the device and/or of the implantation-site.

The concentration-time profile data (to which the PBTK model is fit during a training process) may be predetermined from prior measurements of the concentration of the substance released in tissue, organ, biofluid or excreta compartments of a mammalian system under controlled conditions. In other words, in a training stage the PBTK model can be fit to measurements for the transfer and distribution of a substance in a specific compartment, tissue, organ or biofluid of the mammalian body, or into excreta of the mammal, in order to obtain a value for a number of parameters (biokinetic parameters) within the PBTK model.

Tissue and/or excreta of the mammal may comprise one or more from a group consisting of: peri-implant tissue, tissue and organ compartments, blood, hair/fur, excrement. Said excrement may include urine, faeces, sweat, saliva or other bodily fluids.

Concentration or concentration level may be considered as the abundance of a substance or constituent divided by the total volume of the mixture (the mixture being the substance and the carrier into which it is mixed). The concentration level of the substance in tissue of the mammal may comprise a concentration level of the substance in a specific tissue or biofluid compartment of the mammalian system of interest. Said specific tissues may comprise peri-implant tissue and/or, liver and/or, kidneys and/or lungs, and/or brain, and/or gut and/or skin, and said biofluid comprises serum (blood), and/or urine, and/or sweat. The concentration level of the substance in excreta of the mammal may comprise a concentration level of the substance in one or more of: faeces, urine, sweat, renal excretion, hair, exhalation.

The substance may be selected from the group comprising: a chemical ion, a chemical molecule, a chemical particle, a chemical particulate, a drug, a herbal medicine, a chemical organic compound or a chemical inorganic compound. The substance may be any substance released from any type of medical implant, or any substance present as a result of a chemical reaction released from any type of medical implant with another chemical in the body of a mammal. The medical implant device may be stable or biodegradable.

Optionally, after determining the concentration level, the method further comprises determining a confidence interval associated with the determined concentration level. The confidence interval could also be considered to represent the degree of uncertainty of the time dependent concentration level. The confidence interval represents an interval around the modelled concentration level within which a measurement (of a device having the modelled device-specific parameters) would occur with a given percentage probability. The percentage may be, in particular examples, 95%, 90% or 85%. Said percentage may be set by regulatory bodies, for instance, to set an acceptable level of uncertainly in the modelled concentration levels, in order to depend on the same to support regulatory approval. Confidence levels may be obtained via Monte Carlo simulation techniques.

Optionally, if the maximum of (or other measure derived from) the concentration level exceeds or is equal to the predefined exposure threshold, then the method further comprises adjusting (step 1030) the device-specific data and repeating steps a to d, described above. In other words, if the medical implant device having a certain set of device-specific data is identified as having an unsatisfactory toxicological risk (i.e., potentially harmful to the health of the mammal into which the device is implanted), then the device (and/or its proposed implantation site) is adapted. The device (and/or implantation site) adaptation is represented by an adjustment to the device-specific data characterising the medical device or its behaviour. The new, adjusted device-specific data is then input to the PBTK model, to determine if the adapted device has a reduced maximum concentration level (or other measure derived from the concentration level) that is below the predefined exposure threshold. In this process of iteration and adjustment, a device can be designed with a satisfactory toxicological risk.

As noted above, the device specific data comprises a function describing the rate of release of the substance from the device to local tissue and blood in the blood stream (where blood may refer to blood serum or whole blood), and the function may be obtained through in-vitro measurements of a given device, or as a simulation (i.e., a function of device design parameters and/or implantation site-related parameters). As such, adjusting the device-specific data may comprise constructing a new device an performing an in-vivo test in controlled conditions, in order to obtain a function describing the rate of release of the substance from the device. Alternatively, if the function is representative of a simulation, then one or more of the device design parameters and/or implantation site-related parameters within the function of the simulation may be modified to provide a new function being the adjusted device-specific data.

Adjusting one or more of the device-specific data may use Perturbation Theory Machine Learning (PTML) model for rational selection of device design and/or implantation-site related parameters. This allows the optimisation of the design of the medical implant device. This is particularly suited to the example in which the device-specific data is a function of one or more device design parameters and/or implantation-site related parameters, which simulate the rate of release of a substance from a device at a proposed site of implantation. In particular, PTML can be used to perturb one or more of the device design parameters and/or implantation-site related parameters within the device-specific data so that, after multiple iterations the device design is optimised. PTML may be used to converge the model on a set of device-specific data that minimises the maximum concentration level, or that has a preferred maximum concentration level. The preferred maximum concentration level may be a certain percentage (5%, 10%, 15%, 20% or more) below the predefined exposure threshold for the given tissue and/or excreta. Said percentage may be set by regulatory bodies, for instance.

When using PTML, the method may continue to iterate even if a maximum concentration level is identified below the predefined exposure threshold, in order to converge the model to the set of device-specific data that minimises the maximum concentration level. In this case, there is a method for optimisation of a medical implant device, the device for implant into the body of a mammal and wherein a substance is released from the medical implant device after implantation, and the method may comprise the following steps:
a) inputting device-specific data into a physiologically based toxicokinetic (PBTK) model for the distribution of the substance in the body of the mammal into which the medical implant device is to be implanted, wherein the device-specific data characterises the medical implant device and/or its behaviour at a specific site of implantation;
b) determining, from the PBTK model, a time dependent function for the concentration level of the substance in a tissue, organ, biofluid and/or excreta of the mammal, wherein the substance is a potential toxicant, toxin or drug;
c) determining, using the time dependent function for the concentration level of the substance, the maximum (or peak magnitude) of the concentration level during a predetermined time interval;
d) adjusting the device-specific data and repeating steps a to c, wherein adjusting and repeating uses Perturbation Theory Machine Learning (PTML) model to converge to device-specific data that minimises the maximum (or peak magnitude) concentration level, such that the device-specific data that minimises the maximum (or peak magnitude) concentration level is optimised device-specific data (or an optimised set of device-specific data). The medical implant device may comprise or contain the substance, or a reactant of a chemical reaction forming the substance. The substance (or reactant) is releasable from the medical implant device.

The PBTK model according to this method for optimisation of a medical implant device may be the set of equations according to E1, E2, E3 or E4, as described above, or according to equations E5 and E6 below (wherein, in each case, the PBTK model has been previously trained to fit experimentally measured data). This method of optimisation is particularly suited to the example in which the device specific data is a function of one or more device design parameters and/or implantation-site related parameters, which simulate the rate of release of a substance from a device. The method is for optimising the design of implantable devices, within the device-specific limits that ensure its structural integrity and biofunctionality. As before, the method may be used to optimise the physical or mechanical properties of a device, or its implant location. Thus, the method could be used to identify an optimum implantation site location within a mammal body, even where a known, commercially available device is to be implanted.

In the above method, a further step may be applied, the further step being e) comparing the maximum (or peak magnitude) of the concentration level generated for the optimised set of device-specific data with a predefined exposure threshold for the tissue, organ, biofluid and/or excreta. Even though the device design parameters and/or implantation-site related parameters have been optimised to reduce the concentration level of the substance, the maximum concentration level should still be below the predefined exposure threshold before implantation in a patient. It is envisaged that local minima in the maximum concentration level may be observed upon adjustment of a particular parameter of the device-specific data, and so more than one parameter in the set of device specific data which simulate the rate of release of a substance from a device may need to be adjusted in order to achieve an optimised device having a minimised maximum concentration level that is also below the predefined exposure threshold.

In a specific illustrative example, it is possible to analyse the exchange of nickel ions (Ni) from cardiovascular stents. A simplified version of the model presented in equations E1 (above) is considered here, in which the transport of Ni from the serum (blood) to the local tissue is omitted, and in which the transport of Ni from the serum (blood) to/from skin/fur and feces is omitted. In this simplified model, the equations to be solved are:

$${\dot{M}}_{lt} ( t ) = ( 1 - F ( t ) ) {\dot{M}}_{d} ( t ) - {k}_{lt} ( t ) {M}_{lt} ( t ) , \qquad ( E5 )$$

$${\dot{M}}_{s} ( t ) = F ( t ) {\dot{M}}_{d} ( t ) - ( {k}_{s l} ( t ) + {k}_{s k} ( t ) + {k}_{s l u} ( t ) + {k}_{s g} ( t ) + {k ( t )}_{s b} ) {M}_{s} ( t ) + {k}_{lt} ( t ) {M}_{lt} ( t ) + {k}_{l s} ( t ) {M}_{liv} ( t ) + {k}_{k s} ( t ) {M}_{k} ( t ) + {k}_{l u s} ( t ) {M}_{l u n g} ( t ) + {k}_{g s} ( t ) {M}_{g} ( t ) + {k}_{b s} ( t ) {M}_{b} ( t ) ,$$

$${\dot{M}}_{k} ( t ) = - ( {k}_{k s} ( t ) + {k}_{u} ( t ) ) {M}_{k} ( t ) + {k}_{s k} ( t ) {M}_{s} ( t ) ,$$

$${\dot{M}}_{l i v} ( t ) = - {k}_{ls} ( t ) {M}_{liv} ( t ) + {k}_{s l} ( t ) {M}_{s} ( t ) ,$$

$${\dot{M}}_{l u n g} ( t ) = - {k}_{lus} ( t ) {M}_{lung} ( t ) + {k}_{slu} ( t ) {M}_{s} ( t ) ,$$

$${\dot{M}}_{g} ( t ) = - ( {k}_{g s} ( t ) + {k}_{f} ( t ) ) {M}_{g} ( t ) + {k}_{s g} ( t ) {M}_{s} ( t ) + {k}_{d} ( t ) ,$$

$${\dot{M}}_{b} ( t ) = - {k}_{b s} ( t ) {M}_{b} ( t ) + {k}_{s b} ( t ) {M}_{s} ( t )$$

To calculate the rate of nickel release from the medical device, Saylor et al. (2016) is followed and the following expression for the cumulative amount of nickel release (M_{d}) per surface area (A) is employed which gives the Ni release rate, \dot{M}_{d}(t) of the device, where α is the amount of surface-connected nickel per surface area, _{T} is a characteristic release time, and β is the Higuchi rate constant for elution of embedded nickel normalized by surface area:

$$\frac{{M}_{d} ( t )}{A} = α ( 1 - \frac{8}{{π}^{2}} {e}^{- t / τ} ) + β \sqrt{r} ⇒ \frac{{\dot{M}}_{d} ( t )}{A} = \frac{8 α}{{π}^{2} τ} {e}^{- t / τ} + \frac{β}{2 \sqrt{t}} \qquad ( E6 )$$

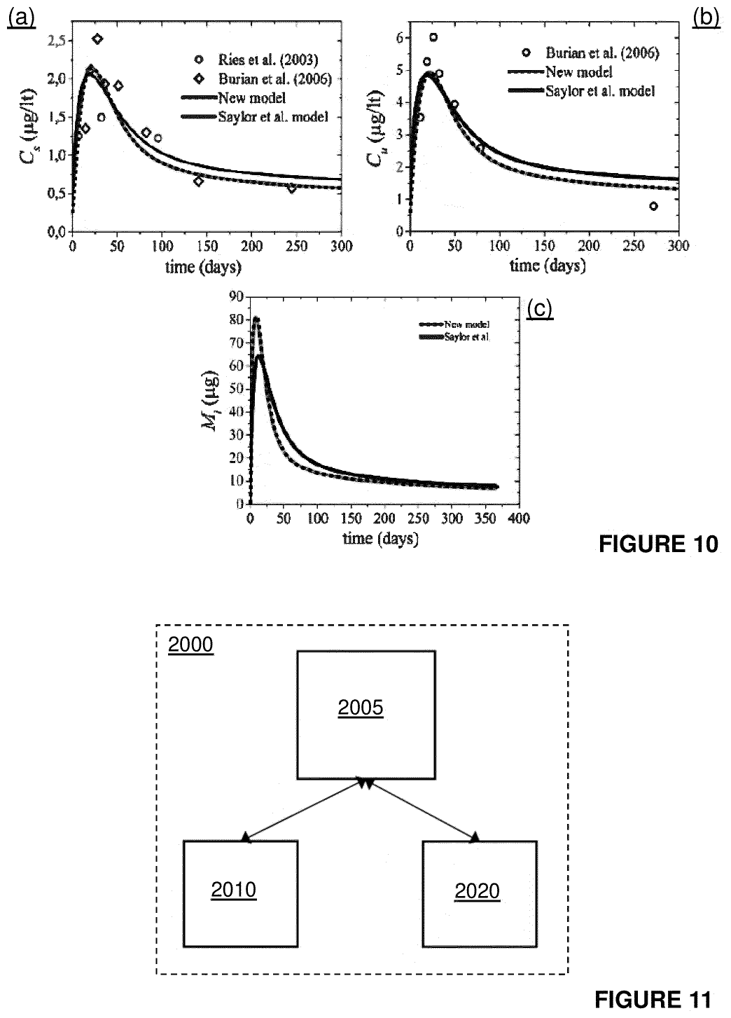

The system of differential equations of the PBTK model can be solved numerically. The device-specific (A, α, β, _{T}) data and kinetic data (Vₛ, Qᵤ, Cₛ₍ₜ₌₀₎, and Cᵤ₍ₜ₌₀₎; via Cₛ=Mₛ/Vₛ and Cᵤ=Mₛkᵤ/Qᵤ) parameter values, as obtained from Saylor et al. (2016) can be used. Fitted values can be obtained for the remaining kinetic parameters. In this example, the PBTK model exhibits quantitative consistency for the time dependent concentration level with reported experimental data for serum and urine levels of nickel (from Burian et al. Int. J. Clin. Pharmacol. Ther. 44 (3), 107-112 (2006) and Ries et al. Am. Heart J. 145 (4), 737-741 (2003)) as well as with the predictions (for serum, urine and local tissue) of the biokinetic model of Saylor et al. (2016). The comparison is shown in FIG. 10, which compares the time dependent concentration level from the model with available experimental data in Burian et al. and Ries et al. and the biokinetic model of Saylor et al. (2016). In particular, FIG. 10(a) shows nickel levels in serum Cₛ, FIG. 10(b) shows nickel levels in urine Cᵤ; and FIG. 10(b) shows nickel levels in tissue local to the device Mₗ. After obtaining the time dependent concentration level from the model as shown in FIG. 10, the peak maximum for the modelled time dependent concentration levels can subsequently be compared to the predefined exposure threshold for nickel in each of the local tissue, the blood (serum) and the urine, in order to assess the toxicological risk (satisfactory or unsatisfactory) of the medical implant device defined by the device-specific data.

A further aspect of the invention is illustrated in FIG. 11. In particular, there is a system for toxicological risk assessment of a medical implant device, the device for implant into the body of a mammal. In other words, the system may be used to implement the method for toxicological risk assessment of a medical implant device described above. The system comprises a device processor (2005); and a non-transitory computer readable medium (2010). The non-transitory computer readable medium (2010) stores instructions that are executable by the device processor to: a) receive device-specific data into a physiologically based toxicokinetic (PBTK) model for the distribution of a substance in the body of the mammal into which the medical implant device is to be implanted, wherein the device-specific data characterises the medical implant device and/or its behaviour at a specific site of implantation; b) determine, from the PBTK model, a time dependent function for the concentration level of the substance in a tissue and/or excreta of the mammal; c) determine, using the time dependent function for the concentration level of the substance, the maximum of the concentration level during a predetermined time interval; d) compare the maximum of the concentration level with a predefined exposure threshold for the tissue, organ, biofluid and/or excreta; wherein if the maximum of the concentration level exceeds or is equal to the predefined exposure threshold, then identify the medical implant device characterised by the device-specific data as having an unsatisfactory toxicological risk; or, if the maximum of the concentration level is less than the predefined exposure threshold then identify the medical implant device characterised by the device-specific data as having a satisfactory toxicological risk.

The characteristics of features and parameters discussed above in relation to the method apply equally to the corresponding features and parameters of the system. The PBTK model may be the set of equations according to E1, E2, E3, E4, E5 or E6 as described above (wherein, in each case, the PBTK model has been previously trained to fit experimentally measured data).

The system may further comprise a data storage (2020), which could be used, for instance, for storage of the received device-specific data, or for storage of any optimised device-specific data.

A system, having the same elements as shown in FIG. 11, may also be used to implement the method for optimisation of a medical implant device, as described above.

In a still further aspect, there is a method of manufacture of a medical implant device, comprising: i) performing in vitro measurements (for instance, following experimental, theoretical or computational tests) on a medical implant device having a set of device design parameters, to obtain device-specific data; and ii) undertaking a toxicological risk assessment of the medical implant device according to the method described above, using the obtained device-specific data; wherein if the maximum of the concentration level exceeds or is equal to the predefined exposure threshold, then the method further comprises adjusting one or more of the device design parameters, and repeating steps i to ii. This method of manufacture is particularly suited to the example in which the device specific data is a function describing the rate of release of the substance from the device to local tissue and blood in the blood stream wherein that function is obtained by in-vitro measurements for a given medical implant device, as described above.

The above description is intended to be complete, although any of a number of acceptable additions, subtractions, or alterations of the described systems and methods used may be made, without departing from the scope of the invention. For example, as illustrated above, some or many of the method steps may be eliminated or performed in a different order. Thus, the above-mentioned description is only intended to provide exemplary purposes, and should not be interpreted, in any way, as limiting the scope of the invention.

1. A method for toxicological risk assessment of a medical implant device, the medical implant device for implant into the body of a mammal and wherein a substance is released from the medical implant device after implantation, comprising:
a) inputting device-specific data into a physiologically based toxicokinetic (PBTK) model for the distribution of the substance in the body of the mammal into which the medical implant device is to be implanted, wherein the device-specific data characterises the medical implant device and/or its behaviour at a specific site of implantation;
b) determining, from the PBTK model, a time dependent function for the concentration level of the substance in a tissue, organ, biofluid and/or excreta of the mammal;
c) determining, using the time dependent function for the concentration level of the substance, the maximum of the concentration level during a predetermined time interval;
d) comparing the maximum of the concentration level with a predefined exposure threshold for the tissue, organ, biofluid and/or excreta;
wherein if the maximum of the concentration level exceeds or is equal to the predefined exposure threshold, then identifying the medical implant device characterised by the device-specific data as having an unsatisfactory toxicological risk; or
if the maximum of the concentration level is less than the predefined exposure threshold then identifying the medical implant device characterised by the device-specific data as having a satisfactory toxicological risk.

2. The method of claim 1, wherein if the maximum of the concentration level exceeds or is equal to the predefined exposure threshold, then the method further comprises adjusting the device-specific data and repeating steps a to d.

3. The method of claim 2, wherein said adjusting one or more of the device-specific data uses Perturbation Theory Machine Learning (PTML) model for rational selection of device design parameters.

4. A method for optimisation of a medical implant device, the medical implant device for implant into the body of a mammal and wherein a substance is released from the medical implant device after implantation, comprising:
a) inputting device-specific data into a physiologically based toxicokinetic (PBTK) model for the distribution of the substance in the body of the mammal into which the medical implant device is to be implanted, wherein the device-specific data characterises the medical implant device and/or its behaviour at a specific site of implantation;
b) determining, from the PBTK model, a time dependent function for the concentration level of a substance in a tissue, organ, biofluid and/or excreta of the mammal;
c) determining, using the time dependent function for the concentration level of the substance, the maximum of the concentration level during a predetermined time interval;
d) adjusting the device-specific data and repeating steps a to c, wherein adjusting and repeating uses a Perturbation Theory Machine Learning (PTML) model to converge to device-specific data that minimises the maximum concentration level, such that the device-specific data that minimises the maximum concentration level is optimised device-specific data.

5. The method of claim 1, wherein the device-specific data comprises a time-dependent function describing the rate of release of the substance from the device to local tissue and to blood.

6. The method of claim 5, wherein the time-dependent function describing the rate of release of the substance from the device to local tissue and blood is obtained by in vitro measurements for a given medical implant device.

7. The method of claim 5, wherein the time-dependent function describing the rate of release of the substance from the device to local tissue and blood is a function of one or more device design parameters and/or one or more implantation site-related parameters.

8. The method of claim 7, wherein the device design parameters comprise one or more parameters selected from a group comprising: total active surface area, method of manufacture, surface treatment and/or modification, geometry, characteristics of the material from which the device is made.

9. The method of claim 7, wherein the implantation site-related data comprise one or more parameters selected from a group comprising: local biomechanical and/or biochemical characteristics, hemorheological properties and hemodynamic parameters that describe the local and peripheral blood flow profile.

10. The method of claim 1, wherein the PBTK model predicts time-dependent substance release from one implant device.

11. The method of claim 10, wherein the PBTK model is described by the set of equations:
$${\dot{M}}_{lt} ( t ) = ( 1 - F ( t ) ) {\dot{M}}_{d} ( t ) - {k}_{lts} ( t ) {M}_{lt} ( t ) + {k}_{slt} ( t ) {M}_{s} ( t ) ,$$
$${\dot{M}}_{s} ( t ) = F ( t ) {\dot{M}}_{d} ( t ) + {k}_{lts} ( t ) {M}_{lt} ( t ) - ( {k}_{s l} ( t ) + {k}_{s k} ( t ) + {k}_{s l u} ( t ) + {k}_{s g} ( t ) + {k}_{s b} ( t ) + {k}_{slt} ( t ) + {k}_{s f} ( t ) ) {M}_{s} ( t ) + {k}_{l s} ( t ) {M}_{l i v} ( t ) + {k}_{k s} ( t ) {M}_{k} ( t ) + {k}_{l u s} ( t ) {M}_{l u n g} ( t ) + {k}_{g s} ( t ) {M}_{g} ( t ) + {k}_{b s} ( t ) {M}_{b} ( t ) ,$$
$${\dot{M}}_{k} ( t ) = - ( {k}_{k s} ( t ) + {k}_{u} ( t ) ) {M}_{k} ( t ) + {k}_{s k} ( t ) {M}_{s} ( t ) ,$$
$${\dot{M}}_{l i v} ( t ) = - {k}_{l s} ( t ) {M}_{l i v} ( t ) + {k}_{s l} ( t ) {M}_{s} ( t ) ,$$
$${\dot{M}}_{l u n g} ( t ) = - {k}_{l u s} ( t ) {M}_{l u n g} ( t ) + {k}_{s l u} ( t ) {M}_{s} ( t ) + {k}_{r e s p} ( t ) ,$$
$${\dot{M}}_{g} ( t ) = - ( {k}_{g s} ( t ) + {k}_{f} ( t ) ) {M}_{g} ( t ) + {k}_{s g} ( t ) {M}_{s} ( t ) + {k}_{d} ( t ) ,$$
$${\dot{M}}_{b} ( t ) = - {k}_{b s} ( t ) {M}_{b} ( t ) + {k}_{s b} ( t ) {M}_{s} ( t ) ,$$
$${\dot{M}}_{u} ( t ) = {C}_{u} ( t ) {Q}_{u} = {k}_{u} ( t ) {M}_{k} ( t ) ,$$
$${\dot{M}}_{f} ( t ) = {C}_{f} ( t ) {Q}_{f} = {k}_{f} ( t ) {M}_{g} ( t ) ,$$
$${\dot{M}}_{s f} ( t ) = {C}_{s f} ( t ) {Q}_{s f} = {k}_{s f} ( t ) {M}_{s} ( t ) ,$$
wherein \dot{M}_{d} is the rate of release of the substance from the medical implant device;
wherein F is a fraction of the substance released directly into the blood and the local tissue from the medical implant device;
wherein k_{d} is rate of dietary absorption; kᵣₑₛₚ is rate of inhalation absorption; k_{f} is rate of elimination from the gut to feces; kᵤ is rate of elimination from kidney to urine; k_{skf} is rate of elimination from the blood to skin-fur/hair; kₗₜₛ and kₛₗₜ are rates of exchange from local tissue to blood and from blood to local tissue, respectively; kₗₛ is rate of exchange from liver to blood; kₛₗ is the rate of exchange from blood to liver; k_{bs} is rate of exchange from brain to blood; k_{sb} is rate of exchange from blood to brain; kₗᵤₛ is rate of exchange from lungs to blood; kₛₗᵤ is rate of exchange from blood to lungs; kₖₛ is rate of exchange from kidney to blood; kₛₖ is rate of exchange from blood to kidney; k_{gs} is rate of exchange from gut to blood; and k_{sg} is rate of exchange from blood to gut;
wherein Mₗₜ, Mₛ, Mₖ, Mₗᵢᵥ, M_{lung}, M_{g}, Mᵤ, M_{f}, M_{sf}, and M_{b} denote the mass of substance in local tissue, blood, kidney, liver, lungs, gut, urine, feces, skin/fur, and brain, respectively;
wherein Cᵤ, C_{f}, and C_{sf}, are substance concentrations in urine, feces, and skin/fur, respectively;
wherein Qᵤ, Q_{f}, and Q_{sf}, are volumetric urine, feces, and sweat outputs.

12. The method of claim 1, wherein the PBTK model predicts time-dependent substance release from two implant devices, the two implant devices implanted simultaneously into the same mammal during at least a portion of the period for which the time-dependent substance release is modelled.

13. The method of claim 12, wherein the two implant devices are implanted in the same site of the mammal body, and wherein the PBTK model is described by the set of equations:
$${\dot{M}}_{lt} ( t ) = ( 1 - {F}_{1} ( t ) ) {\dot{M}}_{d 1} ( t ) + ( 1 - {F}_{2} ( t ) ) {\dot{M}}_{d 2} ( t ) - {k}_{lts} ( t ) {M}_{lt} ( t ) + {k}_{slt} ( t ) {M}_{s} ( t ) ,$$
$${\dot{M}}_{s} ( t ) = {F}_{1} ( t ) {\dot{M}}_{d 1} ( t ) + {F}_{2} ( t ) {\dot{M}}_{d 2} ( t ) + {k}_{lts} ( t ) {M}_{lt} ( t ) - ( {k}_{s l} ( t ) + {k}_{s k} ( t ) + {k}_{s l u} ( t ) + {k}_{s g} ( t ) + {k}_{s b} ( t ) + {k}_{slt} ( t ) + {k}_{s f} ( t ) ) {M}_{s} ( t ) + {k}_{l s} ( t ) {M}_{liv} ( t ) + {k}_{k s} ( t ) {M}_{k} ( t ) + {k}_{l u s} ( t ) {M}_{lung} ( t ) + {k}_{g s} ( t ) {M}_{g} ( t ) + {k}_{b s} ( t ) {M}_{b} ( t ) ,$$
$${\dot{M}}_{k} ( t ) = - ( {k}_{k s} ( t ) + {k}_{u} ( t ) ) {M}_{k} ( t ) + {k}_{s k} ( t ) {M}_{s} ( t ) ,$$
$${\dot{M}}_{liv} ( t ) = - {k}_{ls} ( t ) {M}_{liv} ( t ) + {k}_{s l} ( t ) {M}_{s} ( t ) ,$$
$${\dot{M}}_{l u n g} ( t ) = - {k}_{l u s} ( t ) {M}_{l u n g} ( t ) + {k}_{s l u} ( t ) {M}_{s} ( t ) + {k}_{r e s p} ( t ) ,$$
$${\dot{M}}_{g} ( t ) = - ( {k}_{g s} ( t ) + {k}_{f} ( t ) ) {M}_{g} ( t ) + {k}_{s g} ( t ) {M}_{s} ( t ) + {k}_{d} ( t ) ,$$
$${\dot{M}}_{b} ( t ) = - {k}_{b s} ( t ) {M}_{b} ( t ) + {k}_{s b} ( t ) {M}_{s} ( t ) ,$$
$${\dot{M}}_{u} ( t ) = {C}_{u} ( t ) {Q}_{u} = {k}_{u} ( t ) {M}_{k} ( t ) ,$$
$${\dot{M}}_{f} ( t ) = {C}_{f} ( t ) {Q}_{f} = {k}_{f} ( t ) {M}_{g} ( t ) ,$$
$${\dot{M}}_{s f} ( t ) = {C}_{s f} ( t ) {Q}_{s f} = {k}_{s f} ( t ) {M}_{s} ( t ) ,$$
wherein \dot{M}_{d1} is the rate of release of the substance from a first of the two medical implant devices and \dot{M}_{d2} is the rate of release of the substance from a second of the two medical implant devices;
wherein F₁ is a fraction of the substance released directly into the blood and the local tissue from the first medical implant device and F₂ is a fraction of the substance released directly into the blood and the local tissue from the second medical implant device;
wherein k_{d} is rate of dietary absorption; kᵣₑₛₚ is rate of inhalation absorption; k_{f} is rate of elimination from the gut to feces; kᵤ is rate of elimination from kidney to urine; k_{sf} is rate of elimination from the blood to skin-fur/hair; kₗₜₛ and kₛₗₜ are rates of exchange from local tissue to blood and from blood to local tissue, respectively; kₗₛ is rate of exchange from liver to blood; kₛₗ is the rate of exchange from blood to liver; k_{bs} is rate of exchange from brain to blood; k_{sb} is rate of exchange from blood to brain; kₗᵤₛ is rate of exchange from lungs to blood; kₛₗᵤ is rate of exchange from blood to lungs; kₖₛ is rate of exchange from kidney to blood; kₛₖ is rate of exchange from blood to kidney; k_{gs} is rate of exchange from gut to blood; and k_{sg} is rate of exchange from blood to gut;
wherein Mₗₜ, Mₛ, Mₖ, Mₗᵢᵥ, M_{lung}, M_{g}, Mᵤ, M_{f}, M_{sf}, and M_{b} denote the mass of substance in local tissue, blood, kidney, liver, lungs, gut, urine, feces, skin/fur, and brain, respectively;
wherein Cᵤ, C_{f}, and C_{sf}, are substance concentrations in urine, feces, and skin/fur, respectively;
wherein Qᵤ, Q_{f}, and Q_{sf}, are volumetric urine, feces, and sweat outputs.

14. The method of claim 12, wherein the two implant devices are implanted at different sites of the mammal body, and wherein the PBTK model is described by the set of equations:
$${\dot{M}}_{lt 1} ( t ) = ( 1 - {F}_{1} ( t ) ) {\dot{M}}_{d 1} ( t ) - {k}_{lt 1 s} ( t ) {M}_{lt 1} ( t ) + {k}_{slt 1} ( t ) {M}_{s} ( t ) ,$$
$${\dot{M}}_{lt 2} ( t ) = ( 1 - {F}_{2} ( t ) ) {\dot{M}}_{d 2} ( t ) - {k}_{lt 2 s} ( t ) {M}_{lt 2} ( t ) + {k}_{slt 2} ( t ) {M}_{s} ( t ) ,$$
$${\dot{M}}_{s} ( t ) = {F}_{1} ( t ) {\dot{M}}_{d 1} ( t ) + {F}_{2} ( t ) {\dot{M}}_{d 2} ( t ) + {k}_{lt 1 s} ( t ) {M}_{lt 1} ( t ) + {k}_{lt 2 s} ( t ) {M}_{lt 2} ( t ) - ( {k}_{s l} ( t ) + {k}_{s k} ( t ) + {k}_{slu} ( t ) + {k}_{s g} ( t ) + {k}_{s b} ( t ) + {k}_{slt 1} ( t ) + {k}_{slt 2} ( t ) + {k}_{s f} ( t ) ) {M}_{s} ( t ) + {k}_{ls} ( t ) {M}_{liv} ( t ) + {k}_{k s} ( t ) {M}_{k} ( t ) + {k}_{lus} ( t ) {M}_{lung} ( t ) + {k}_{g s} ( t ) {M}_{g} ( t ) + {k}_{b s} ( t ) {M}_{b} ( t ) ,$$
$${\dot{M}}_{k} ( t ) = - ( {k}_{k s} ( t ) + {k}_{u} ( t ) ) {M}_{k} ( t ) + {k}_{s k} ( t ) {M}_{s} ( t ) ,$$
$${\dot{M}}_{liv} ( t ) = - {k}_{ls} ( t ) {M}_{liv} ( t ) + {k}_{s l} ( t ) {M}_{s} ( t ) ,$$
$${\dot{M}}_{lung} ( t ) = - {k}_{lus} ( t ) {M}_{lung} ( t ) + {k}_{slu} ( t ) {M}_{s} ( t ) + {k}_{r e s p} ( t ) ,$$
$${\dot{M}}_{g} ( t ) = - ( {k}_{g s} ( t ) + {k}_{f} ( t ) ) {M}_{g} ( t ) + {k}_{s g} ( t ) {M}_{s} ( t ) + {k}_{d} ( t ) ,$$
$${\dot{M}}_{b} ( t ) = - {k}_{b s} ( t ) {M}_{b} ( t ) + {k}_{s b} ( t ) {M}_{s} ( t ) ,$$
$${\dot{M}}_{u} ( t ) = {C}_{u} ( t ) {Q}_{u} = {k}_{u} ( t ) {M}_{k} ( t ) ,$$
$${\dot{M}}_{f} ( t ) = {C}_{f} ( t ) {Q}_{f} = {k}_{f} ( t ) {M}_{g} ( t ) ,$$
$${\dot{M}}_{s f} ( t ) = {C}_{s f} ( t ) {Q}_{s f} = {k}_{s f} ( t ) {M}_{s} ( t ) ,$$
wherein \dot{M}_{d1} is the rate of release of the substance from a first of the two medical implant devices and \dot{M}_{d2} is the rate of release of the substance from a second of the two medical implant devices;
wherein F₁ is a fraction of the substance released directly into the blood and the local tissue from the first medical implant device and F₂ is a fraction of the substance released directly into the blood and the local tissue from the second medical implant device;
wherein k_{d} is rate of dietary absorption; kᵣₑₛₚ is rate of inhalation absorption; k_{f} is rate of elimination from the gut to feces; kᵤ is rate of elimination from kidney to urine; k_{sf} is rate of elimination from the blood to skin-fur/hair; kₗₜ₁ₛ and kₗₜ₂ₛ are the rates of exchange from the first local tissue to blood (serum) and second local tissue to blood (serum), respectively; kₛₗₜ₁ and kₛₗₜ₂ are the rates of exchange from blood (serum) to the first local tissue, and from blood (serum) to the second local tissue, respectively; kₗₛ is rate of exchange from liver to blood; kₛₗ is the rate for exchange from blood to liver; k_{bs} is rate of exchange from brain to blood; k_{sb} is rate of exchange from blood to brain; kₗᵤₛ is rate of exchange from lungs to blood; kₛₗᵤ is rate of exchange from blood to lungs; kₖₛ is rate of exchange from kidney to blood; kₛₖ is rate of exchange from blood to kidney; k_{gs} is rate of exchange from gut to blood; and k_{sg} is rate of exchange from blood to gut;
wherein Mₗₜ₁, Mₗₜ₂, Mₛ, Mₖ, Mₗᵢᵥ, M_{lung}, M_{g}, Mᵤ, M_{f}, M_{sf}, and M_{b} denote the mass of substance in local tissues 1 and 2, blood, kidney, liver, lungs, gut, urine, feces, skin/fur, and brain, respectively;
wherein Cᵤ, C_{f}, and C_{sf}, are substance concentrations in urine, feces, and skin/fur, respectively;
wherein Qᵤ, Q_{f}, and Q_{sf}, are volumetric urine, feces, and sweat outputs.

15. The method of claim 1, further comprising, prior to inputting device-specific data into the PBTK model, training the PBTK model.

16. The method of claim 1, wherein training the PBTK model comprises fitting the PBTK model to in vivo experimental data, in order to determine values for kinetic parameters within the PBTK model.

17. The method of claim 1, wherein the concentration level of the substance in a mammalian tissue comprises a concentration level of the substance in a specific tissue, organ or biofluid compartment of the mammalian system of interest.

18. The method of claim 1, wherein the concentration level of the substance in excreta comprises a concentration level of the substance in one or more of: faeces, urine, sweat, renal excretion, hair, exhalation.

19. The method of claim 1, wherein the substance is selected from the group comprising: a chemical ion, a chemical particle, a chemical particulate, a chemical molecule, a drug, a herbal medicine, a chemical organic compound, a chemical inorganic compound.

20. The method of claim 1, further comprising, after determining the time dependent function for the concentration level of the substance in the tissue, organ, biofluid and/or excreta of the mammal, determining a confidence interval associated with the time dependent function for the concentration level of the substance in the tissue, organ, biofluid and/or excreta of the mammal.

21. A system for toxicological risk assessment of a medical implant device, the device for implant into the body of a mammal, comprising:
a device processor;
a non-transitory computer readable medium storing instruction that are executable by the device processor to perform the steps of the method according to claim 1.

22. A method of manufacture of a medical implant device, comprising:
i) performing in vitro measurements on a medical implant device having a set of device design parameters, to obtain device-specific data; and
ii) undertaking a toxicological risk assessment of the medical implant device according to claim 1, using the obtained device-specific data;
wherein if the maximum of the concentration level exceeds or is equal to the predefined exposure threshold, then the method further comprises adjusting the device-specific data, and repeating steps i to ii; and
iii) once the adjusted device-specific data result in a toxicological risk assessment in which the maximum of the concentration level is less than the predefined exposure threshold, then manufacturing the implant device with said adjusted device-specific data.

23. A medical implant device designed and manufactured by the method of claim 22.

24. A method of manufacture of a medical implant device, comprising:
i) undertaking optimisation of a medical implant device according to claim 4, to obtain optimised device-specific data;
(ii) comparing the maximum of the concentration level generated for the optimised device-specific data with a predefined exposure threshold for the tissue, organ, biofluid and/or excreta;
wherein if the maximum of the concentration level is less than the predefined exposure threshold, then
(iii) manufacturing the implant device with said optimised device-specific data.

25. A medical implant device designed and manufactured by the method of claim 24.
